SEQUENCE LISTING (42 sequences)

SEQ ID NO: 1 - DNA, 340 bp, unidentified
  misc_feature 1..340: note = SV40 Promoter
  source 1..340: mol_type = unassigned DNA
gctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 2 - DNA, 171 bp, unidentified
  misc_feature 1..171: note = delta3 SV40 Promoter
  source 1..171: mol_type = unassigned DNA
ctgtggaatgtgtgtcagttagtcccgcccctaactccgcccagttccgcccattctccgccccatggctgactaattttttttatttatgcagaggccgaggccgcctcggcctctgagctattccagaagtagtgaggaggcttttttggaggcctaggcttttgcaaa

SEQ ID NO: 3 - DNA, 1650 bp, unidentified
  misc_feature 1..1650: note = CMV Promoter
  source 1..1650: mol_type = unassigned DNA
cgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacattgcagaatcaccaccgagaggataatgatcaaggagtacggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctgtaaggccgtgacaacatacacgaggattggataggcctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacg

SEQ ID NO: 4 - DNA, 2742 bp, unidentified
  misc_feature 1..2742: note = Hamster b-actin Promoter
  source 1..2742: mol_type = unassigned DNA
agtcctcattgacaggcagtattcgctctgccccacccccacctgtgaattgcagggctggcaggtcctcaggcagctggcaaaccgcctgaacaactgagagatacagggccagggccagggcagtcccgtcccccggaggcagggaggggacgtgctgggaaagttctctctctcaggcccaggttggtgactgcagaaggcttctgtcaaatctcttttgtgggaaccacagagtagccctgaacgtgggggtgtgcttccagtatactctggggtcaccctttccatactggaggcctctgcaacttcaaaatgctctgctaccaacctagcacaaggaagttggtccagcctccccacgcagggccactgctgcagtccatatatggactaagccttccttggtttcaacacctacactcactgagcccctactatgtgtatgcagagccgagacaggccctgagcatctcatctgaagcacccttcttgcctaaattcagttttctgtcactttctcccaggaggtgtgtgtccctctaagctaagccaggggtccctcacccctgccccactcccatccctagtgtaggtatcagctgaagagcttcctgagcagaacactcttgggtgctgacattttgataaataggcccatgtttaggagagcaggggtccgggggcgggagatcttctctggtggattgagggctccaagaactactctttgagcacgctgcccctcccagagtccccacagcctccagatggactagaacacagttcggctgtggctgcacataactaacagaggatagatggtgggtcccagcccaacagtgcctggcaatcacccagagccaccagctaacggccttggcttagttttttgcctgggtgtgatcaggcagccctccaaaactgcccggactccatgacaagttttgcttgttctatagagcacagttcctttctaggtctggggcaagggacatcgggagacatcttcctgcaacagctccagtcactggaccaccaggctcgccctgtctttggtgtgtggccctgagtctcctaagtggcccaaacctgtgaagacccctccaaccacagttttgcttctaaattgtaccccaacacacctagcaaattgaaaccccaccagaagtcccccagatctggctttccggctattgctggcaagggggagtgactcccggcccattcaatccaggccccgcgtgttcctcaaacaagaagccacgtaaacataaaccgagcctccatgctgacccttgcccatcgaggtactcaatgttcacgtgatatccacacccagagggtcctggggtgggtgcatgagccccagaatgcaggcttgataaccgagaccctgaatcgggcagtgtccacaagggcggaggccagtcatgcatgttcgggcctatggggccagcacccaacgccaaaactctccatcctcttcctcaatctcgctttctctctctctctctttttttttttttattttttttttttgcaaaaggaggggagagggggtaaaaaaatgctgcactgtgcggctaggccggtgagtgagcggcgcggagccaatcagcgctcgccgttccgaaagttgccttttatggctcgagtggccgctgtggcgtcctataaaacccggcggcgcaacgcgcagccactgtcgagtccgcgtccacccgcgagcacaggcctttcgcagctctttcttcgccgctccacacccgccaccaggtaagcagggacaacaggcccagccggccacagccctcccgtgggcagtgaccgcgctgcagggtcgcgggggacactcggcgcggacaccggggaaggctggagggtggtgccgggccgcggagcggacactttcagatccaactttcagtccagggtgtagaccctttacagccgcattgccacggtgtagacaccggtggacccgctctggctcagagcacgcggcttgggggaacccattagggtcgcagtgtgggcgctatgagagccgatgcagctttcgggtgttgaaccgtatctgcccaccttggggggaggacacaaggtcgggagccaaacgccacgatcatgccttggtggcccatgggtctttgtctaaaccggtttgcccatttggcttgccgggcgggcgggcgcggcgggcccggctcggccgggtgggggctgggttgccactgcgcttgcgcgctctatggctgggtattggggcgcgtgcacgctggggagggagcccttcctcttccccctctcccaagttaaacttgcgcgtgcgtattgagacttggagcgcggccaccggggttgggcgagggcggggccgttgtccggaaggggcggggtcgcagcggcttcggggcgcctgctcgcgcttcctgctgggtgtggtcgcctcccgcgcgcgcactagccgcccgccggcggggcgaaggcggggcttgcgcccgtttggggagggggcggaggcctggcttcctgccgtggggccgcctccggaccagcgtttgcctcttatggtaataacgcggccggcctgggcttcctttgtcccctgagtttgggcgcgcgccccctggcggcccgaggccgcggcttgccggaagtgggcagggcggcagcggctgcgcctagtggcccgctagtgaccgcgaccctcttttgtgccctgatatagttcgccgg

SEQ ID NO: 5 - DNA, 1122 bp, unidentified
  misc_feature 1..1122: note = Hamster Glutamine synthetase coding sequence
  source 1..1122: mol_type = unassigned DNA
atggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggaatttcaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaa

SEQ ID NO: 6 - protein, 373 aa, unidentified
  REGION 1..373: note = Hamster Glutamine synthetase amino acid sequence
MATSASSHLNKNIKQMYLCLPQGEKVQAMYIWVDGTGEGLRCKTRTLDCEPKCVEELPEWNFDGSSTFQSEGSNSDMYLSPVAMFRDPFRRDPNKLVFCEVFKYNRKPAETNLRHSCKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWVARFILHRVCEDFGVIATFDPKPIPGNWNGAGCHTNFSTKAMREENGLKHIEEAIEKLSKRHRYHIRAYDPKGGLDNARRLTGFHETSNINDFSAGVANRSASIRIPRTVGQEKKGYFEDRRPSANCDPFAVTEAIVRTCLLNETGDEPFQYKN

SEQ ID NO: 7 - DNA, 186 bp, unidentified
  misc_feature 1..186: note = CD52 FLARE Reporter coding sequence
  source 1..186: mol_type = unassigned DNA
ttggagcgcttcctcttcctcctactcaccatcagcctcctcgttttggtacaaatacaaaccggactctccggacaaaacgacaccagccaaaccagcagcccctcagcatccagcaacataagcggaggcattttccttttcttcgtcgccaacgccataatccacctcttctgcttcagttga

SEQ ID NO: 8 - protein, 61 aa, unidentified
  REGION 1..61: note = CD52 FLARE Reporter amino acid sequence
LERFLFLLLTISLLVLVQIQTGLSGQNDTSQTSSPSASSNISGGIFLFFVANAIIHLFCFS

SEQ ID NO: 9 - DNA, 1194 bp, synthetic construct
  misc_feature 1..1194: note = Synthetic: Ubiquitous chromatin opening element (A2UCOE)
  source 1..1194: mol_type = other DNA
ggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgccttagtgggggggaaggttctagaaaagcggcggcagcggctctagcggcagtagcagcagcgccgggtcccgtgcggaggtgctcctcgcagagttgtttctccagcagcggcagttctcactacagcgccaggacgagtccggttcgtgttcgtccgcggagatctctctcatctcgctcggctgcgggaaatcgggctgaagcgactgagt

SEQ ID NO: 10 - DNA, 232 bp, unidentified
  misc_feature 1..232: note = Chicken hyper-sensitivity site 4 (CHS4) core insulator
  source 1..232: mol_type = unassigned DNA
gagggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaa

SEQ ID NO: 11 - DNA, 705 bp, synthetic construct
  misc_feature 1..705: note = Synthetic: Anti-Factor Bb antibody light chain coding sequence
  source 1..705: mol_type = other DNA
atgagagttccagctcagctgctgggactgctgctgttgtggttgcctggcgctagatgcgacatccagatgacccagtctccatccacactgtccgcctctgtgggcgacagagtgaccatcacatgcaaggcctctcaggatgtgggcaccgccgttgcttggtatcagcagaagcctggcaaggcccctaagctgctgatctactgggcctccaccagacataccggcgtgcccgatagattctccggctctggctctggcaccgactttaccctgacaatcagctccctgcaggccgaggatttcgccgtgtacttctgccaccagcacagcagcaaccctctgacctttggccagggcaccaagctggaaatcaagcggacagtcgccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 12 - protein, 214 aa, synthetic construct
  REGION 1..214: note = Synthetic: Anti-Factor Bb antibody light chain amino acid sequence (without signal sequence)
DIQMTQSPSTLSASVGDRVTITCKASQDVGTAVAWYQQKPGKAPKLLIYWASTRHTGVPDRFSGSGSGTDFTLTISSLQAEDFAVYFCHQHSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1386 bp, synthetic construct
  misc_feature 1..1386: note = Synthetic: Anti-Factor Bb antibody heavy chain coding sequence
  source 1..1386: mol_type = other DNA
atgggatggtctttgatcctgctgttcctggtggccgtggctaccagagtgcattctgaggtgcagctggtggaatctggcggcggacttgtgaaacctggcggctctctgagactgtcttgtgccgcttctggcttcaccttctccaactacgccatgtcctgggtccgacaggctcctggcaaaagactggaatgggtcgccaccatctccaaccggggctcctacacctactatcccgactccgtgaagggcagattcaccatcagccgggacaacgccaagaactccctgtacctgcagatgaacagcctgagagccgaggacaccgctctgtactactgcgccagagaacggcccatggattattggggccagggcacactggtcaccgtgtcctctgcttctaccaagggacccagcgtgttccctctggctccttgctccagatccacctccgagtctacagctgctctgggctgcctggtcaaggactactttcctgagcctgtgaccgtgtcttggaactctggcgctctgacatccggcgtgcacacctttccagctgtgctgcaatcctccggcctgtactctctgtcctccgtcgtgaccgtgccttctagctctctgggcaccaagacctacacctgtaatgtggaccacaagccttccaacaccaaggtggacaagcgcgtggaatctaagtacggccctccttgtcctccatgtccagctccagagtttgaaggcggcccttccgtgttcctgtttcctccaaagcctaaggacaccctgatgatctctcggacccctgaagtgacctgcgtggtggtggatgtgtcccaagaggatcccgaggtgcagttcaattggtacgtggacggcgtggaagtgcacaatgccaagaccaagcctagagaggaacagttcaactccacctatagagtggtgtccgtgctgaccgtgctgcaccaggattggctgaacggcaaagagtacaagtgcaaggtgtccaacaagggcctgcctagctccatcgagaaaaccatcagcaaggccaagggccagcctcgggaacctcaggtttacaccctgcctccaagccaagaggaaatgaccaagaaccaggtgtccctgacctgcctcgtgaagggattctacccctccgatatcgccgtggaatgggagtctaatggacagcccgagaacaactacaagacaacccctcctgtgctggactccgacggcagcttcttcctgtattctcgcctgaccgtggacaagtccagatggcaagagggcaacgtgttctcctgcagcgtgctgcatgaggccctgcactctcactacacccagaagtccctgtctctgtccctgggcaaatga

SEQ ID NO: 14 - protein, 442 aa, synthetic construct
  REGION 1..442: note = Synthetic: Anti-Factor Bb antibody heavy chain amino acid sequence (without signal sequence)
EVQLVESGGGLVKPGGSLRLSCAASGFTFSNYAMSWVRQAPGKRLEWVATISNRGSYTYYPDSVKGRFTISRDNAKNSLYLQMNSLRAEDTALYYCARERPMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - DNA, 702 bp, synthetic construct
  misc_feature 1..702: note = Synthetic: Anti-CD40L antibody light chain coding sequence
  source 1..702: mol_type = other DNA
atgggctggtcctgcatcatcctgtttctggtggctaccgctaccggcgtgcactccgatatcgtgatgacccagtctcctagcttcctgtccgcctctgtgggcgacagagtgacaattacctgcaaggcctcctccaacctgggccatgctgtggcttggtatcagcagaagcctggcaagtcccctaagctgctgatctactccgcctccaacagatacacaggcgtgcccgacagattctccggctctggctctggcacagactttaccctgacaatctccagcctgcagcctgaggacttcgccgactacttctgccagcagtacgacgactacccctacacctttggcggaggcaccaagctggaaatcaagcggacagtggccgctccttccgtgttcatcttcccaccttccgacgagcagctgaagtctggcaccgcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctga

SEQ ID NO: 16 - protein, 214 aa, synthetic construct
  REGION 1..214: note = Synthetic: Anti-CD40L antibody light chain amino acid sequence (without signal sequence)
DIVMTQSPSFLSASVGDRVTITCKASSNLGHAVAWYQQKPGKSPKLLIYSASNRYTGVPDRFSGSGSGTDFTLTISSLQPEDFADYFCQQYDDYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 1410 bp, synthetic construct
  misc_feature 1..1410: note = Synthetic: Anti-CD40L antibody heavy chain coding sequence
  source 1..1410: mol_type = other DNA
atgggctggtcctgcatcatcctgtttctggtggctaccgctaccggcgtgcactctgaagttcagctgcaagagtctggccctggcctggtcaagccttccgaaacactgtctctgacctgcaccgtgtccggcgactctatcaccaacggcttttggatctggatccggaagcctcctggcaacaagctcgagtatatgggctacatctcctactccggctccacctactacaaccccagcctgaagtcccggatctccatctccagagacacctccaagaaccagttctccctgaagctgtcctccgtgaccgctgctgatacaggcgtgtactactgcgcctaccggtcctatggcagaaccccttactacttcgactactggggccagggcaccacactgacagtgtcctctgcttccaccaagggacccagcgttttccctctggctccatcctccaagtctacctctggcggaacagctgctctgggctgtctggtcaaggactacttccctgagcctgtgaccgtgtcctggaattctggcgctctgacatctggcgtgcacacctttccagctgtgctgcagtcctccggcctgtactctctgtcctctgtcgtgaccgtgccttccagctctctgggaacccagacctacatctgcaatgtgaaccacaagcctagcaacaccaaggtggacaagaaggccgagcctaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcaccgggatccagaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcgccgtgtccaacaaggccctgcctgctcctatcgaaaagaccatctccaaggctaagggccagcctcgggaacctcaggtgtacacattgcctccatctcgggacgagctgaccaagaatcaggtgtccctgacctgtctcgtgaagggcttctacccctccgatatcgccgtggaatgggagtccaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacccagaagtccctgtctctgtcccctggcaaatga

SEQ ID NO: 18 - protein, 450 aa, synthetic construct
  REGION 1..450: note = Synthetic: Anti-CD40L antibody heavy chain amino acid sequence (without signal sequence)
EVQLQESGPGLVKPSETLSLTCTVSGDSITNGFWIWIRKPPGNKLEYMGYISYSGSTYYNPSLKSRISISRDTSKNQFSLKLSSVTAADTGVYYCAYRSYGRTPYYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKAEPKSCDKTHTCPPCPAPELLGGPSVFLFPPKPKDTLMISRTPEVTCVVVDVSHRDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 717 bp, synthetic construct
  misc_feature 1..717: note = Synthetic: Anti- a-synuclein/IGF1R antibody light chain coding sequence
  source 1..717: mol_type = other DNA
atgggctggtcctgcatcattctgtttctggtggctaccgctaccggcgtgcactccgatatcgtgatgacccagacacctctgagcctgtctgtgacacctggccagcctgcctccatctcctgcagatcttctcagtccctggtgcactccaacggcaacacctacctgcactggtatctgcagaagcccggacagtctccccagctgctgatctacaaggtgtccaacagattctctggcgtgcccgacagattcagcggctctggctctggcaccgacttcaccctgaagatctctagagtggaagccgaggacgtgggcgtgtactactgctctcagtctacccacgtgccaagaacctttggccagggcaccaagctggaaatcaagagaaccgtggccgctccttccgtgttcatcttcccaccatctgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggactctacctactctctgtcctccacactgaccctgtccaaggccgactacgagaagcacaaggtgtacgcctgtgaagtgacccaccagggcctgtctagccctgtgaccaagtctttcaaccggggcgagtgctga

SEQ ID NO: 20 - protein, 219 aa, synthetic construct
  REGION 1..219: note = Synthetic: Anti- a-synuclein/IGF1R antibody light chain amino acid sequence (without signal sequence)
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - DNA, 1395 bp, synthetic construct
  misc_feature 1..1395: note = Synthetic: Anti- a-synuclein/IGF1R antibody heavy chain 1 coding sequence
  source 1..1395: mol_type = other DNA
atgggctggtcctgcatcattctgtttctggtggctaccgctaccggcgtgcactctcaggttcagttggttcagtctggcgccgaagtgaagaaacctggcgcttctgtgaaggtgtcctgcaaggcctctggctacacctttaccaactacctgatcgagtgggtccgacaggctcctggacaaggactggaatggatgggcgtgatcaatcctggctctggcggcaccaactacaacgagaagttcaagggcagagtgaccatgaccagagacacctccatctccaccgcctacatggaactgtcccggctgagatctgacgacaccgccgtgtactactgcgcctctggcaactacgatacctattggggccagggcacactggtcaccgtgtcctctgcttctaccaagggaccctctgtgttccctctggctccttccagcaagtccacatctggtggaaccgctgctctgggctgcctcgtgaaggattactttcctgagcctgtgaccgtgtcttggaactctggtgctctgacctctggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaactgctcggcggtccctccgttttcctgtttccacctaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacacactgcctccatctagggacgagctgaccaagaatcaggtgtccctgtggtgcctggtcaagggcttctacccttccgatatcgccgtggaatgggagtccaatggccagcctgagaacaactacaagacaacccctcctgtgctggactccgacggctcattcttcctgtactccaagctgacagtggacaagtctcggtggcagcagggcaacgtgttctcttgtagtgtgctgcacgaggccctgcacaaccactacacacagaagtccctgtctctgtcccctggcaaatga

SEQ ID NO: 22 - protein, 445 aa, synthetic construct
  REGION 1..445: note = Synthetic: Anti- a-synuclein/IGF1R antibody heavy chain 1 amino acid sequence (without signal sequence)
QVQLVQSGAEVKKPGASVKVSCKASGYTFTNYLIEWVRQAPGQGLEWMGVINPGSGGTNYNEKFKGRVTMTRDTSISTAYMELSRLRSDDTAVYYCASGNYDTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVLHEALHNHYTQKSLSLSPGK

SEQ ID NO: 23 - DNA, 2193 bp, synthetic construct
  misc_feature 1..2193: note = Synthetic: Anti- a-synuclein/IGF1R antibody heavy chain 2 coding sequence
  source 1..2193: mol_type = other DNA
atgggctggtcctgcatcattctgtttctggtggctaccgctaccggcgtgcactctcaggttcagttggttcagtctggcgccgaagtgaagaaacctggcgcttctgtgaaggtgtcctgcaaggcctctggctacacctttaccaactacctgatcgagtgggtccgacaggctcctggacaaggactggaatggatgggcgtgatcaatcctggctctggcggcaccaactacaacgagaagttcaagggcagagtgaccatgaccagagacacctccatctccaccgcctacatggaactgtcccggctgagatctgacgacaccgccgtgtactactgcgcctctggcaactacgatacctattggggccagggcacactggtcaccgtgtcctctgcttctaccaagggaccctctgtgttccctctggctccttccagcaagtccacatctggtggaaccgctgctctgggctgcctcgtgaaggattactttcctgagcctgtgaccgtgtcttggaactctggtgctctgacctctggcgtgcacacctttccagctgtgctgcagtcctctggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaactgctcggcggtccctccgttttcctgtttccacctaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacaactccacctacagagtggtgtccgtgctgaccgtgctgcaccaggattggctgaacggcaaagagtacaagtgcaaggtgtccaacaaggcactgcccgctcctatcgaaaagaccatctccaaggctaagggccagcctcgggaacctcaggtttacacactgcctccatctagggacgagctgaccaagaaccaggtgtccctgtcttgtgccgtgaagggcttctacccttccgatatcgccgtggaatgggagtccaatggccagcctgagaacaactacaagacaacccctcctgtgctggactccgacggctcattcttcctggtgtccaagctgacagtggacaagtccagatggcagcagggcaacgtgttctcctgtagcgtgctgcatgaggccctgcacaaccactacacacagaagtctctgtctctgagccctggcaaaggtggcggaggatctggcggaggtggaagcggcggaggcggatctcaatctgttctgactcagcctccttccgcttctggcacacctggccagagagtgacaatctcttgcaccggctccagctccaacatcggctccaacgatgtgtcctggtatcagcagctgcctggcacagcccctaaactgctgatctacgccaatgtgaacagaccctccggcgtgcccgatagattctctggctctaagtctggcaccagcgccagcctggctatctctggactgagatccgaggacgaggccgactactattgtggcacttgggccggatctctgaacgcctatgtgttcggctgtggcacaaagctgactgttcttggaggcggtggtagtggtggtggtggttctggtggtggcggaagcggaggtggtggatctgaagttcagctgttggaatctggcggcggacttgttcagcctggtggctctttgagactgtcttgcgctgcctccggcttcaccttctcctcttacgacatgtcctgggttcgacaagccccaggcaagtgtctggaatgggtttccgccatctcctacgataacgccaacacctactacgccgacagcgtgaagggaagattcaccatctctcgggacaacagcaagaacaccctgtacctgcagatgaactccctgagagctgaggatacagctgtctactactgtgccaagggcgtgctgacaacactgatgaattggttcgactactggggacagggaaccctcgtgacagtgtcctcttga

SEQ ID NO: 24 - protein, 711 aa, synthetic construct
  REGION 1..711: note = Synthetic: Anti- a-synuclein/IGF1R antibody heavy chain 2 amino acid sequence (without signal sequence)
QVQLVQSGAEVKKPGASVKVSCKASGYTFTNYLIEWVRQAPGQGLEWMGVINPGSGGTNYNEKFKGRVTMTRDTSISTAYMELSRLRSDDTAVYYCASGNYDTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPGKGGGGSGGGGSGGGGSQSVLTQPPSASGTPGQRVTISCTGSSSNIGSNDVSWYQQLPGTAPKLLIYANVNRPSGVPDRFSGSKSGTSASLAISGLRSEDEADYYCGTWAGSLNAYVFGCGTKLTVLGGGGSGGGGSGGGGSGGGGSEVQLLESGGGLVQPGGSLRLSCAASGFTFSSYDMSWVRQAPGKCLEWVSAISYDNANTYYADSVKGRFTISRDNSKNTLYLQMNSLRAEDTAVYYCAKGVLTTLMNWFDYWGQGTLVTVSS

SEQ ID NO: 25 - DNA, 238 bp, synthetic construct
  misc_feature 1..238: note = Synthetic: 5 ITR
  source 1..238: mol_type = other DNA
ccctagaaagataatcatattgtgacgtacgttaaagataatcatgcgtaaaattgacgcatgtgttttatcggtctgtatatcgaggtttatttattaatttgaatagatattaagttttattatatttacacttacatactaataataaattcaacaaacaatttatttatgtttatttatttattaaaaaaaaacaaaaactcaaaatttcttctataaagtaacaaaactttta

SEQ ID NO: 26 - DNA, 309 bp, synthetic construct
  misc_feature 1..309: note = Synthetic: 3 ITR and internal domain
  source 1..309: mol_type = other DNA
gatatctataacaagaaaatatatatataataagttatcacgtaagtagaacatgaaataacaatataattatcgtatgagttaaatcttaaaagtcacgtaaaagataatcatgcgtcattttgactcacgcggtcgttatagttcaaaatcagtgacacttaccgcattgacaagcacgcctcacgggagctccaagcggcgactgagatgtcctaaatgcacagcgacggattcgcgctatttagaaagagagagcaatatttcaagaatgcatgcgtcaattttacgcagactatctttctaggg

SEQ ID NO: 27 - DNA, 20 bp, synthetic construct
  misc_feature 1..20: note = Synthetic: Heavy chain forward primer
  source 1..20: mol_type = other DNA
ctccaaagcctaaggacacc

SEQ ID NO: 28 - DNA, 20 bp, synthetic construct
  misc_feature 1..20: note = Synthetic: Heavy chain reverse primer
  source 1..20: mol_type = other DNA
cgtaccaattgaactgcacc

SEQ ID NO: 29 - DNA, 25 bp, synthetic construct
  misc_feature 1..25: note = Synthetic: Heavy chain probe
  source 1..25: mol_type = other DNA
cggacccctgaagtgacctgcgtgg

SEQ ID NO: 30 - DNA, 19 bp, synthetic construct
  misc_feature 1..19: note = Synthetic: Light chain forward primer
  source 1..19: mol_type = other DNA
cccaagagtctgtgaccga

SEQ ID NO: 31 - DNA, 20 bp, synthetic construct
  misc_feature 1..20: note = Synthetic: Light chain reverse primer
  source 1..20: mol_type = other DNA
gtacaccttgtgcttctcgt

SEQ ID NO: 32 - DNA, 25 bp, synthetic construct
  misc_feature 1..25: note = Synthetic: Light chain probe
  source 1..25: mol_type = other DNA
ccacactgaccctgtccaaggccga

SEQ ID NO: 33 - DNA, 20 bp, synthetic construct
  misc_feature 1..20: note = Synthetic: GAPDH Forward Primer
  source 1..20: mol_type = other DNA
tcaagaaggtggtgaagcag

SEQ ID NO: 34 - DNA, 20 bp, synthetic construct
  misc_feature 1..20: note = Synthetic: GAPDH Reverse Primer
  source 1..20: mol_type = other DNA
caaaggtggaagagtgggag

SEQ ID NO: 35 - DNA, 25 bp, synthetic construct
  misc_feature 1..25: note = Synthetic: GAPDH Probe
  source 1..25: mol_type = other DNA
tgggctacaccgaggaccaggttgt

SEQ ID NO: 36 - protein, 594 aa, unidentified
  REGION 1..594: note = Cabbage looper moth transposase sequence 1
MGSSLDDEHILSALLQSDDELVGEDSDSEISDHVSEDDVQSDTEEAFIDEVHEVQPTSSGSEILDEQNVIEQPGSSLASNKILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVRGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSREKFMRNLYMSLTSSFMRKRLEAPTLKRYLRDNISNILPNEVPGTSDDSTEEPVTKKRTYCTYCPSKIRRKANASCKKCKKVICREHNIDMCQSCF

SEQ ID NO: 37 - protein, 594 aa, unidentified
  REGION 1..594: note = Cabbage looper moth transposase sequence 2
MGSSLDDEHILSALLQSDDELVGEDSDSEVSDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTSATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRVYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPKEVPGTSDDSTEEPVMKKRTYCTYCPSKIRRKANASCKKCKKVICREHNIDMCQSCF

SEQ ID NO: 38 - protein, 373 aa, Homo sapiens
  source 1..373: note = Human glutamine synthase
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 39 - DNA, 705 bp, synthetic construct
  source 1..705: mol_type = other DNA
atgggctggtcctgcatcatcctgtttctggtggctaccgctaccggcgtgcactctgatgtgcagatgacccagtctccttccagcctgtctgcctctgtgggcgacagagtgaccatcacctgtcaggccagccagtctatctactccttcctgtcctggtatcagcagaagcccggccaggctcctaagctgctgatctacgctgcctccgacctggaatctggcgtgccctctagattctccggctctggctctggcaccgactttaccctgacaatctccagcctgcagcctgaggacttcgccacctactactgccagtccaactacatcatcgactacggcgcctttggccagggcaccaaggtggtcatcaagagaacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctatagcctgtcctccacactgaccctgtccaaggccgactacgagaagcacaaggtgtacgcctgcgaagtgacccatcagggcctgtctagtcccgtgaccaagtctttcaaccggggcgagtgttga

SEQ ID NO: 40 - protein, 215 aa, synthetic construct
DVQMTQSPSSLSASVGDRVTITCQASQSIYSFLSWYQQKPGQAPKLLIYAASDLESGVPSRFSGSGSGTDFTLTISSLQPEDFATYYCQSNYIIDYGAFGQGTKVVIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - DNA, 1398 bp, synthetic construct
  source 1..1398: mol_type = other DNA
atgggctggtcctgcatcatcctgtttctggtggctaccgctaccggcgtgcactctgaagtgcagctggaagaatctggcggcagactggtgcagcctggcacatctctgagactgtcttgtgccgtgtccggcttctccctgtcctcctacgatatgtcctgggtccgacaggctcctggcaaaggactggaatgggtgtccaccatctacatcggcggaacaaccgcctacgcctcctggcctaagggcagattcaccatctccaagaccaactccaagaacaccctgtacctgcagatgaactccctgagagccgaggataccgccgtgtacttctgcgctagactgcagggcgccaactactacaactctctggctctgtggggccagggcacactggttacagtgtcctccgcttccaccaagggaccctctgtgtttcctctggctccttgctccagatccacctccgagtctacagctgctctgggctgcctggtcaaggactactttcctgagcctgtgaccgtgtcctggaactctggcgctctgacatctggcgtgcacacctttccagctgtgctgcagtcctccggcctgtactctctgtcctctgtcgtgaccgtgccttcctctagcctgggcaccaagacctacacctgtaatgtggaccacaagccttccaacaccaaggtggacaagcgcgtggaatctaagtacggccctccttgtcctccatgtcctgctccagaagctgctggcggcccatcagtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgctaagaccaagcctagagaggaacagttcaactccacctacagagtggtgtccgtgctgaccgtgctgcatcaggattggctgaacggcaaagagtacaagtgcaaggtgtccaacaagggcctgccttccagcatcgaaaagaccatcagcaaggccaagggccagcctagggaaccccaggtttacaccctgcctccaagccaagaggaaatgaccaagaaccaggtgtccctgacctgcctcgtgaagggcttctacccttccgatatcgccgtggaatgggagagcaatggccagcctgagaacaactacaagacaacccctcctgtgctggactccgacggcagcttcttcctgtattcccgcctgaccgtggacaagtccagatggcaagagggcaacgtgttctcctgctccgtgatgcacgaggccctgcacaatcactacacccagaagtccctgtctctgtccctgggctaa

SEQ ID NO: 42 - protein, 446 aa, synthetic construct
EVQLEESGGRLVQPGTSLRLSCAVSGFSLSSYDMSWVRQAPGKGLEWVSTIYIGGTTAYASWPKGRFTISKTNSKNTLYLQMNSLRAEDTAVYFCARLQGANYYNSLALWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2026/0347790 A1
(43) Pub. Date: Nov. 5, 2026

(54) EXPRESSION CONSTRUCTS FOR PRODUCING RECOMBINANT ANTIBODIES

(71) Applicant: SANOFI, Paris (FR)

(72) Inventor: John SCARCELLI, Holliston, MA (US)

(73) Assignee: SANOFI, Paris (FR)

(21) Appl. No.: 19/657,440

(22) Filed: Apr. 24, 2026

Related U.S. Application Data
(60) Provisional application No. 63/795,080, filed on Apr. 25, 2025.

(30) Foreign Application Priority Data
Apr. 28, 2025 (EP) 25305607.1

(51) Int. Cl. C12N 5/071; C07K 14/705; C07K 16/00; C12N 15/85; C12P 21/00

(52) U.S. Cl. CPC C12N 5/0682; C07K 14/70592; C07K 16/00; C12N 15/85; C12P 21/00; C07K 2317/14; C12N 2510/00

(57) ABSTRACT

The present disclosure provides novel semi-targeted polycistronic transposon-based expression constructs and methods of producing recombinant antibodies therefrom.

### CROSS-REFERENCE TO RELATED APPLICATION

This application claims priority from U.S. Patent Application No. 63/795,080, filed Apr. 25, 2025, and European Priority Application No. 25305607.1 filed Apr. 28, 2025, the disclosures of each of which are incorporated herein by reference in their entirety.

### SEQUENCE LISTING

The application contains a Sequence Listing which has been submitted electronically in .XML format and is hereby incorporated by reference in its entirety. Said .XML copy, created on Apr. 23, 2026, is named “022548.WO067.xml” and is 76,914 bytes in size. The sequence listing contained in this .XML file is part of the specification and is hereby incorporated by reference herein in its entirety.

### BACKGROUND OF THE INVENTION

Generation of stable recombinant cell lines producing high titers of monoclonal antibodies is an important aspect of biopharmaceutical production. Antibodies are assembled from at least two different polypeptides (heavy chain and light chain). Ensuring stable expression levels and proper relative ratios of the various antibody chains in producing host cells is critical to large-scale antibody production. It is challenging to achieve high expression levels of recombinant proteins, especially proteins assembled from different polypeptide chains, as increased genetic loads can strain the host cell's translational machinery (Codarri et al., Nature (2022) 610(7930):161-72). Maintenance of stable long-term expression of recombinant proteins presents an additional challenge because host cells may experience reduced productivity or loss of the transgene over time (Peltret et al., J Biotech. (2024) 389:30-42). Given the prominent role of antibodies as therapeutics for numerous diseases, there remains a need for antibody-producing cell lines with improved expression efficiency and long-term stability.

### SUMMARY OF THE INVENTION

The present disclosure provides an expression construct for expressing a recombinant antibody in a mammalian host cell, comprising a polycistronic expression cassette flanked by a pair of inverted terminal repeats (ITR) recognizable by a transposase, wherein the expression cassette comprises, from 5′ to 3′, a first cistron encoding a light chain of the antibody and comprising a first promoter, a second cistron encoding a selection marker and comprising a second promoter, and a third cistron encoding a heavy chain of the antibody and comprising a third promoter, wherein the first and third promoters are identical or are of substantially equivalent transcriptional strength in a mammalian host cell.

In some embodiments, the recombinant antibody is monospecific.

In some embodiments, the transposase is a cabbage looper moth transposase.

In some embodiments, the expression construct further comprises a first insulator (i.e., spacer) sequence between the ITR at the 5′ end of the expression cassette (5′ ITR) and the first cistron and/or a second insulator sequence between the third cistron and the ITR at the 3′ end of the expression cassette (3′ ITR).

In some embodiments, each of the first and third promoters is a cytomegalovirus (CMV) promoter, a simian virus 40 (SV40) promoter, a truncated SV40 promoter (Δ3SV40), or a hamster β-actin (haBA) promoter. In further embodiments, both the first and third promoters are CMV promoters.

In some embodiments, the selection marker is glutamine synthetase (GS). In certain embodiments, the GS is a hamster GS comprising SEQ ID NO:6 or an amino acid sequence at least about 85% identical thereto.

In some embodiments, at least one of the cistrons encodes more than one polypeptide. For example, the third cistron may comprise a coding sequence for a detectable marker upstream of a coding sequence for the heavy chain, wherein the coding sequence for the detectable marker has a non-ATG start codon and the coding sequence for the heavy chain has an ATG start codon, and the third promoter directs expression of both the detectable marker and the heavy chain.

In some embodiments, the detectable marker is a cell surface protein. In further embodiments, the cell surface protein is a human CD52 comprising SEQ ID NO:8 or an amino acid sequence at least about 85% identical thereto.

In some embodiments, the polycistronic expression cassette comprises a ubiquitous chromatin opening element (UCOE). In further embodiments, the UCOE is placed between the second and third cistrons, and/or comprises SEQ ID NO:9 or a nucleotide sequence at least about 90% identical thereto.

In some embodiments, each of the first and third promoters is a CMV promoter, and the second cistron comprises a coding sequence for a hamster GS under control of a Δ3SV40 promoter, and/or the third cistron comprises a coding sequence for a human CD52 upstream of a coding sequence for the heavy chain, wherein the coding sequence for the human CD52 has a non-ATG start codon and the coding sequence for the heavy chain has an ATG start codon, and the third promoter directs expression of both the human CD52 and the heavy chain. In further embodiments, the CMV promoter comprises SEQ ID NO:3 or a nucleotide sequence at least about 90% identical thereto, the hamster GS comprises SEQ ID NO:6 or an amino acid sequence at least about 90% identical thereto, the Δ3SV40 promoter comprises SEQ ID NO:2 or a nucleotide sequence at least about 90% identical thereto, and/or the human CD52 comprises SEQ ID NO:8 or an amino acid sequence at least about 90% identical thereto.

In another aspect, the present disclosure provides a method of generating a mammalian host cell that produces a recombinant antibody, comprising: introducing into a population of mammalian cells (i) the expression construct described herein and (ii) the transposase or an expression vector for the transposase, culturing the cells in a selection medium that allows survival of cells that express the selection marker, and isolating a surviving cell, thereby generating a mammalian host cell that produces the recombinant antibody. In further embodiments, the selection marker is GS (e.g., human or hamster GS) and the selection medium does not comprise glutamine. In certain embodiments, the expression construct expresses a detectable marker and the isolating step comprises selecting a surviving cell that expresses the detectable marker (e.g., a human CD52), by, e.g., flow cytometry.

In another aspect, the present disclosure provides a mammalian host cell that comprises in its genome the expression construct described herein, or that is produced by the producing method described herein. In some embodiments, the host cell is a CHO cell. In further embodiments, the host cell (e.g., CHO cell) comprises a null mutation in an endogenous GS gene.

In another aspect, the present disclosure provides a method of producing a recombinant antibody, comprising culturing the mammalian host cell described herein under conditions that allow expression of the antibody, and isolating the antibody from the culture.

Other features, objectives, and advantages of the invention are apparent in the detailed description that follows. It should be understood, however, that the detailed description, while indicating embodiments and aspects of the invention, is given by way of illustration only, not limitation. Various changes and modifications within the scope of the invention will become apparent to those skilled in the art from the detailed description.

### BRIEF DESCRIPTION OF THE FIGURES

FIGS. 1A and 1B are schematic diagrams showing the orientation topology alignment of various monoclonal antibody double-gene expression vectors (#10, #12, #13, #14, #14b, #14c, #14d, #15, #16, #17, #18, #19, #20, #21, and #22). Expression elements were inserted into the vector backbone at one of three positions indicated at the top of each figure. CMV: human cytomegalovirus promoter. haBA: hamster β-actin promoter. SV40: full-length simian virus 40 promoter. Δ3SV40: truncated version (Δ3) of simian virus 40 promoter. GS: hamster glutamine synthetase. UCOE: ubiquitous chromatin opening element (A2UCOE). CD52 human CD52 cell surface receptor encoded by a sequence that with an attenuated start codon (UUG). ITR: inverted terminal repeats. CHS4: chicken hypersensitivity site 4 core insulator. HC: heavy chain. LC: light chain.

FIG. 1C is a map for a plasmid containing the vector #17 configuration, illustrated with the coding sequences for the light and heavy chains of an anti-Factor Bb antibody.

FIG. 1D is a schematic diagram illustrating a transposon with the vector #17 configuration. This transposon includes inverted terminal repeats (ITRs) recognizable by a transposase, a strong promoter (black arrow) driving light chain (LC) expression, a weak promoter for the selectable marker (lighter color arrow), and a strong promoter (black arrow) driving heavy chain (HC) expression. This vector topology generates high titer pools and clones compared to other vector topologies.

FIG. 1E is a schematic diagram illustrating the topology of transposons for expression of a monoclonal antibody (top), a bispecific antibody with a common light chain (middle), or a 4-chain multispecific antibody (bottom). These diagrams are designed according to FIG. 1D.

FIG. 2 is a line graph showing the pool viability profiles of vectors #13-16 during selection. Pools were transfected in duplicates and all pool selection was completed in 14 days except pools with #14b, which displayed delayed pool recovery and pool selection was completed on day 21.

FIG. 3 is a set of bar graphs showing the titer (top) and heavy chain (HC) and light chain (LC) transcript expression (bottom) of fifteen piggyBac® pools expressing anti-Factor Bb. In the bottom graph, dark color bars indicate LC transcript (bar on left for each group of two bars), and light color bars (bar on right for each group of two bars) indicate HC transcript. PB SGV: piggyBac® single gene vector control. All transcript levels were normalized to the control GAPDH transcript levels.

FIG. 4 is a line graph showing the pool viability profiles of vectors #17-22 during selection, with vector #14b as a comparator. Pools were transfected in duplicates and all pool selection was completed in 25 days except #21, which finished selection in 28 days.

FIG. 5 is a bar graph showing the stable pool Ambr® 15 fed-batch titer production after pool selection for vectors #13-16. Day 7 and 12 conditioned media were harvested and analyzed for pool productivity.

FIG. 6 is a bar graph showing the stable pool reporter expression after pool selection for the indicated vectors. Cells were incubated with FITC-conjugated anti-human CD52 antibody for analysis. A mock control pool not expressing the reporter was used as a negative control. The reporter expression is represented as a measure of the geometric mean fluorescence intensity (GMFI) and quantified in relative fluorescence units (RFU).

FIG. 7 is a bar graph showing the vector #14b clone batch titer productivities after clone expansion, with vector #14b presort pool productivities (14b pool 1 and 14b pool 2) shown as a comparator. The vector #14b pools were combined and sorted via reporter expression at the top 10%, loaded onto the Beacon® Optofluidic System for clone generation, screened (1301) for the secreted antibody target and the top clones (75) were exported for clone expansion. The top surviving clones (53) are shown and were evaluated for batch titer productivities. All batch clone collections were performed on day 7.

FIG. 8 is a line graph showing the vector #14b Ambr® 15 fed-batch clone titer productivities after clone expansion, with vector #14b presort pool productivities (dotted lines) shown as a comparator. The top 28 clones are shown and were evaluated for fed-batch titer productivities. All batch clone collection harvests were performed on days 3, 4, 7 and 10 and titer analysis was performed on the Cedex® Bio HT Analyzer.

FIG. 9 is a set of bar graphs showing antibody titers from clarified day 7 harvests of a terminal batch culture of clones expressing an anti-Factor Bb antibody (top) or an anti-CD40L antibody (bottom) through a vector with the #14b or #17 topology.

FIG. 10 is a set of bar graphs showing the transcript levels (top) and copy number (bottom) of the #14b clones. haGS: exogenous Cricetulus griseus glutamine synthetase expression.

FIG. 11A is a bar graph showing the antibody titers from clarified day 7 harvests of batch culture of pools expressing an anti-CD40L antibody through a vector with the #14b or #17 topology.

FIG. 11B is a bar graph showing the antibody titers obtained for an anti-CD40L antibody and a bispecific anti-α-synuclein/anti-IGF1R antibody when expressed from two vectors: vector #14b expressing a light chain, a GS selectable marker, and a heavy chain fused to an scFv; and vector #17 expressing a second light chain, a GS selectable marker, and a heavy chain. These two vectors were transfected in equal proportions. Antibody titers were analyzed after batch and simplified fed batch expression.

FIG. 11C is a schematic diagram illustrating three different transposon topologies for expressing the α-synuclein/IGF1R bispecific antibody from two different expression vectors. These diagrams are designed according to FIG. 1A.

FIG. 12A is a bar graph showing the antibody titers from clarified day 7 harvests of batch culture of pools expressing an anti-IL-1R3 antibody through a vector with the #14b or #17 topology.

FIG. 12B is a bar graph showing antibody titers from harvests of a terminal batch culture of clones expressing an anti-IL-1R3 antibody through a vector with the #17 topology.

### DETAILED DESCRIPTION OF THE INVENTION

The present disclosure is based on the discovery of a novel configuration of polycistronic, transposon-based expression vectors for the co-expression of multiple transgenes such as the heavy chain (HC) and light chain (LC) of an antibody and a selection marker. The expression vector comprises a polycistronic expression cassette flanked by ITRs recognizable by a transposase. Three or more transgenes are arranged in a specific order within the expression cassette, leading to consistently high and stable antibody titers.

The present vectors are semi-targeted. When co-introduced into a mammalian host cell (e.g., a CHO cell) with a transposase, the expression cassette is excised from the carrier construct (i.e., an expression construct) and stably integrates into the host cell's genome at highly expressed TTAA motifs, which are associated with transcriptionally active regions. This semi-targeted integration leads to enhanced protein expression and greater cell line stability. This gene-delivery system can accommodate large DNA cargoes (>200 kb) during transfection, making it particularly advantageous for constructing complex expression systems.

The present polycistronic transposon-based vector system eliminates the need for the use of multiple vectors for expressing multiple proteins or polypeptides, and reduces the total transgene copy number. These features foster a more controlled protein expression environment, which is crucial for ensuring consistent expression, quality, and functionality of therapeutic proteins. By reducing variability in transgene expression and controlling relative expression levels of multiple polypeptides, the current vector system also minimizes the risk of protein misfolding and improves overall protein quality. For example, due to the novel configuration of the vector, the light chain of an antibody can be produced at a higher level of the heavy chain of the antibody, thereby facilitating proper folding and assembly of a functional antibody.

### I. Polycistronic Expression Vectors

The present polycistronic expression vectors (i.e., expression constructs) comprise a polycistronic expression cassette that is flanked by ITRs recognizable by a transposase such that the expression cassette becomes a transposon unit. The expression cassette can be carried within a circular or linear DNA vector, e.g., a plasmid vector. Components of the expression cassette are described in detail below.

### A. Inverted Terminal Repeats

The present expression cassette comprises an ITR at each of its 5′ and 3′ ends. The sequence of the ITR is recognized by a transposase from a transposition system such as the piggyBac® (PB) system, the Sleeping Beauty (SB) system, or the Tol2 system (see, e.g., Sandoval-Villegas et al., Int JMol Sci. (2021) 22(10):5084). In some embodiments, elements from the PB system are used, wherein the transposase is derived from an insect, such as cabbage looper moth (Trichoplusia ni). An exemplary amino acid sequence of Trichoplusia ni transposase is available at UniProt (Accession No. Q283G1) and shown below:

| (SEQ ID NO: 36) | |
|---|---|
| 1 | MGSSLDDEHI LSALLQSDDE LVGEDSDSEI SDHVSEDDVQ SDTEEAFIDE |
| 51 | VHEVQPTSSG SEILDEQNVI EQPGSSLASN KILTLPQRTI RGKNKHCWST |
| 101 | SKSTRRSRVS ALNIVRSQRG PTRMCRNIYD PLLCFKLFFT DEIISEIVKW |
| 151 | TNAEISLKRR ESMTGATFRD TNEDEIYAFF GILVMTAVRK DNHMSTDDLF |
| 201 | DRSLSMVYVS VMSRDRFDFL IRCLRMDDKS IRPTLRENDV FTPVRKIWDL |
| 251 | FIHQCIQNYT PGAHLTIDEQ LLGFRGRCPF RMYIPNKPSK YGIKILMMCD |
| 301 | SGTKYMINGM PYLGRGTQTN GVPLGEYYVK ELSKPVRGSC RNITCDNWFT |
| 351 | SIPLAKNLLQ EPYKLTIVGT VRSNKREIPE VLKNSRSRPV GTSMFCFDGP |
| 401 | LTLVSYKPKP AKMVYLLSSC DEDASINEST GKPQMVMYYN QTKGGVDTLD |
| 451 | QMCSVMTCSR KTNRWPMALL YGMINIACIN SFIIYSHNVS SKGEKVQSRE |
| 501 | KFMRNLYMSL TSSFMRKRLE APTLKRYLRD NISNILPNEV PGTSDDSTEE |
| 551 | PVTKKRTYCT YCPSKIRRKA NASCKKCKKV ICREHNIDMC QSCF |

Variants of the above sequence may also be used, such as those at least about 90% (e.g., at least about 91, about 92, about 93, about 94, about 95, about 96, about 97, about 98, or about 99%) identical thereto. An exemplary variant sequence is SEQ ID NO:37. A 5′ ITR sequence that is recognized by this transposase may comprise SEQ ID NO:25, or a nucleotide sequence at least about 90% (e.g., at least about 91, about 92, about 93, about 94, about 95, about 96, about 97, about 98, or about 99%) identical thereto, whereas a 3′ ITR sequence that is recognized by this transposase may comprise SEQ ID NO:26, or a nucleotide sequence at least about 90% (e.g., at least about 91, about 92, about 93, about 94, about 95, about 96, about 97, about 98, or about 99%) identical thereto.

As used herein, the percent identity of two amino acid sequences or of two nucleotide sequences may be obtained by, e.g., BLAST® using default parameters (available at the U.S. National Library of Medicine's National Center for Biotechnology Information website). In some embodiments, the length of a reference sequence aligned for comparison purposes is at least about 60%, at least about 70%, at least about 80%, or at least about 90%) of the reference sequence.

In some embodiments, the polycistronic expression cassette comprises one or more insulator sequences between the 5′ ITR and the first cistron and/or between the last cistron and the 3′ ITR. Insulator sequences may inhibit negative interference from neighboring genomic regions on the stable and long-term expression of the expression cassette. For example, the insulator is a chicken hypersensitivity site 4 (CHS4) core insulator, such as one comprising SEQ ID NO:10 or a nucleotide sequence at least about 85% (e.g., at least about 86, about 87, about 88, about 89, about 90, about 91, about 92, about 93, about 94, about 95, about 96, about 97, about 98, or about 99%) identical thereto.

### B. Cistron Arrangements

The present expression cassette comprises three or more cistrons, each comprising its own promoter for directing transcription of one or more transgenes within the cistron. For example, the expression cassette comprises at least three, at least four, at least five, or at least six cistrons. The present inventors have surprisingly discovered that cistrons placed near the 5′ and 3′ ends of the polycistronic expression cassette are transcribed at higher levels than a cistron in between, with the cistron near the 5′ end being expressed at the highest level. Thus, depending on the desired relative expression levels of the transgenes, the transgenes can be placed in different locations within the polycistronic expression cassette.

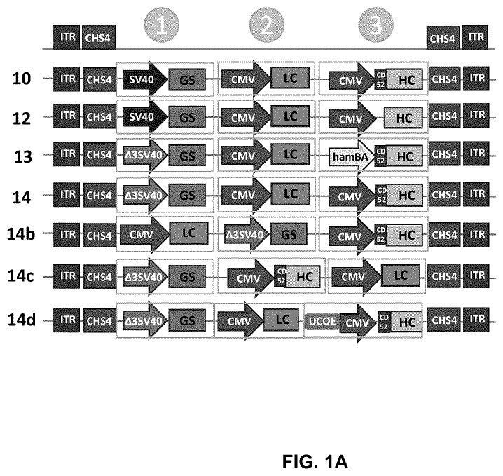

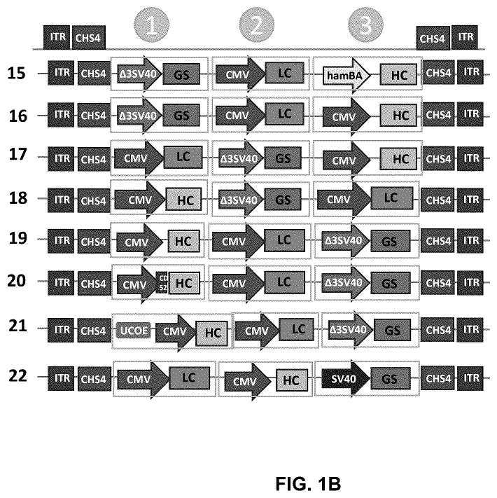

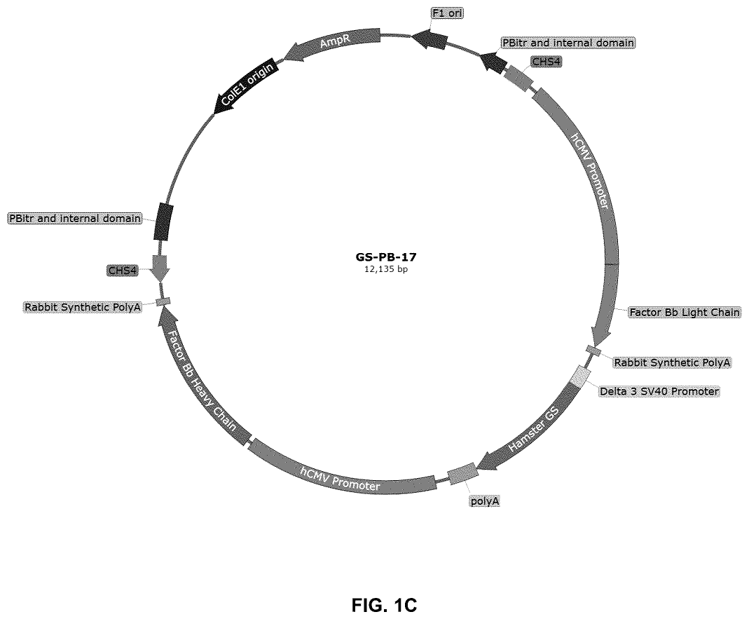

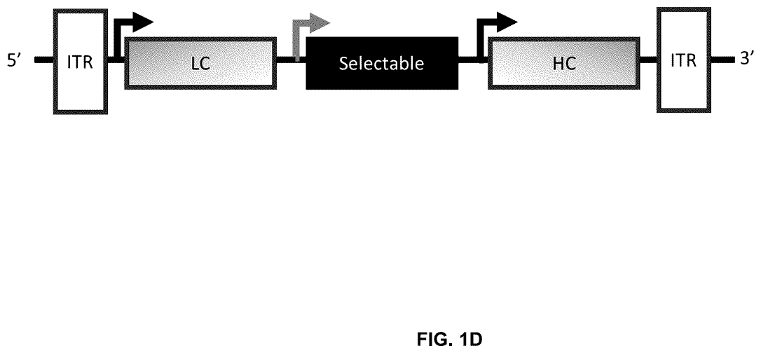

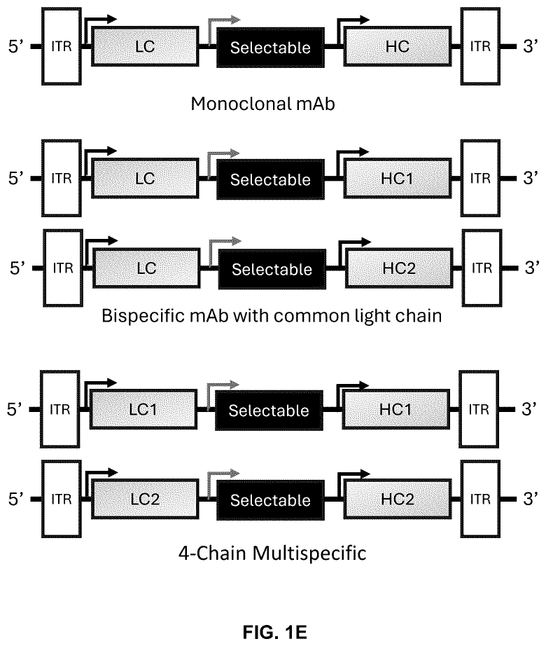

In some embodiments, the expression cassette contains three cistrons, in order of, from 5′ to 3′, first cistron, second cistron, and third cistron, each cistron comprising its own promoter, with a transgene desired to be expressed at the highest level be placed within the first cistron, a transgene desired to be expressed at the second highest level be placed within the third cistron, and the transgene that can be expressed at the lowest level be placed within the second cistron. For example, to promote protein folding and assembly of an antibody, the light chain is usually preferred to be expressed at an excess amount relative to the heavy chain; as such, the light chain coding sequence can be placed in the first cistron, and the heavy chain coding sequence can be placed in the third cistron, with a coding sequence for a selection marker of genomic integration being placed in the second cistron (see, e.g., FIG. 1D).

In the polycistronic expression cassette, each cistron comprises its own promoter. The promoter is operative in a mammalian cell and may be constitutive or inducible. The promoter may be, for example, a human cytomegalovirus (CMV) promoter or a truncated version thereof (e.g., miniCMV), a hamster β-actin (haBA) promoter, a simian virus 40 (SV40) promoter, a truncated SV40 promoter (Δ1SV40, Δ2SV40, or Δ3SV40), a chicken (3-actin (CBA) promoter, a synapsin promoter, an EF1α promoter, a human ubiquitin C promoter, a GRK1 promoter, a rhodopsin promoter, a PGK promoter, a GAPDH promoter, a Q-actin promoter, an Hspa5 promoter, a calreticulin promoter, a farnesyltransferase inhibitor-induced promoter, or a PDIA3 promoter.

In some embodiments, the SV40 promoter comprises SEQ ID NO:1 or a nucleotide sequence at least about 85% (e.g., at least about 86, about 87, about 88, about 89, about 90, about 91, about 92, about 93, about 94, about 95, about 96, about 97, about 98, or about 99%) identical thereto.

In some embodiments, the Δ3SV40 promoter comprises SEQ ID NO:2 or a nucleotide sequence at least about 85% (e.g., at least about 86, about 87, about 88, about 89, about 90, about 91, about 92, about 93, about 94, about 95, about 96, about 97, about 98, or about 99%) identical thereto.

In some embodiments, the CMV promoter comprises SEQ ID NO:3 or a nucleotide sequence at least about 85% (e.g., at least about 86, about 87, about 88, about 89, about 90, about 91, about 92, about 93, about 94, about 95, about 96, about 97, about 98, or about 99%) identical thereto.

In some embodiments, the haBA promoter comprises SEQ ID NO.4 or a nucleotide sequence at least about 85% (e.g., at least about 86, about 87, about 88, about 89, about 90, about 91, about 92, about 93, about 94, about 95, about 96, about 97, about 98, or about 99%) identical thereto.

In some embodiments, two or more cistrons within the polycistronic expression cassette use an identical promoter or promoters of comparable strengths. As used herein, promoters having “comparable strengths” or “substantially equivalent transcriptional activity” refers to promoters that drives expression levels within a 2-fold or 3-fold difference when measured using standard reporter gene assays (e.g., luciferase assay) under identical conditions in the same mammalian cell type. For example, in a tricistronic expression cassette, the first cistron and the third cistron use a CMV promoter, and the second cistron comprises a different promoter such as a SV40 promoter or a weaker promoter (e.g., Δ3SV40 promoter). Alternatively, the first and third cistrons use different promoters of comparable strengths, and the second cistron uses a weaker promoter. Promoters of comparable strengths are, for example, a CMV, a hamster β-actin promoter, a EF1α promoter, and a PGK promoter. Promoters weaker than these promoters, which may be used to drive expression of the middle cistron, include, without limitation, a SV40, a truncated Δ1-3 SV40 promoter, or a miniCMV promoter.

In some embodiments, the expression cassette comprises additional transcription regulatory elements that enhance or maintains expression of the integrated cassette. For example, one or more cistrons in the polycistronic vector comprise a ubiquitous chromatin opening element (UCOE), e.g., upstream of a promoter. In some embodiments, the UCOE is derived from human HNRPA2B1-CBX3 (A2UCOE). In further embodiments, the UCOE comprises SEQ ID NO:9 or a nucleotide sequence at least about 85% (e.g., at least about 86, about 87, about 88, about 89, about 90, about 91, about 92, about 93, about 94, about 95, about 96, about 97, about 98, or about 99%) identical thereto.

Each cistron may comprise a polyadenylation (poly(A)) signal sequence. For example, the polyadenylation signal sequence may be a naturally occurring poly(A) signal sequence or a synthetic one (e.g., a rabbit synthetic poly(A) sequence, or an SV40 poly(A) sequence).

### C. Transgene Sequences

In the polycistronic expression cassette, each cistron may express one or more polypeptides under the transcription regulation of a single promoter. When more than one polypeptide is expressed from a single promoter, an internal ribosome entry site (IRES) may be placed between the coding sequences of the polypeptides. Alternatively, a coding sequence for a self-cleaving peptide (e.g., F2A, E2A, P2A, and T2A) may be placed in frame between coding sequences of the polypeptides. In another alternative, one or more coding sequences for an intein may be included such that the coding sequences are translated into polypeptide sequences that are joined by the intein sequence before excision of the intein. In yet another alternative, the coding sequences of the two polypeptides may use start codons of different strengths; for example, the polypeptide desired to be expressed at a higher level can use the standard ATG start codon and the polypeptide that can be expressed at a lower level can use a non-standard start codon such as TTG or CTG.

The transgenes in the polycistronic expression cassette may encode different proteins and/or different polypeptide chains of the same protein. In some embodiments, the transgenes, such as the transgene in the first and last cistrons in the cassette, may encode the light and heavy chains of an antibody or an antigen-binding fragment thereof (e.g., scFv, Fab, or F(ab′)₂).

In some embodiments, the antibody is monospecific. As used herein, “monospecific” refers to an antibody in which every antigen-binding site recognizes the same epitope; all paratopes are identical, so the molecule can engage only one epitopic target. In other embodiments, the antibody is multispecific; for example, the antibody is bispecific and comprises two different polypeptide chains—two identical light chains, and two identical heavy chains whose C-termini are fused to an scFv against another antigen. In another version of a bispecific antibody, the antibody comprises three different polypeptide chains—two identical light chains, one regular heavy chain, and a second heavy chain whose C-terminus is fused to an scFv against another antigen. In yet another version of a bispecific antibody, the antibody comprises four different polypeptide chains—two different heavy chains and two different light chains and the two heavy chains may comprise charge mutations and/or knob-in-hole mutations to promote the desired heterodimeric pairing. In further alternatives, a multispecific antibody may be a cross-over dual variable IgG (CODV) antibody. In another alternative, a multispecific antibody may further comprise a cytokine (e.g., interleukin-2 (IL-2), interleukin-12 (IL-12), or tumor necrosis factor (TNF)) fused to the C-terminus of the antibody.

In some embodiments, a multispecific antibody may be expressed from one or more different expression vectors. For example, a first expression vector may comprise transgenes encoding a first light chain and a first heavy chain of the multispecific antibody, while a second expression vector may comprise transgenes encoding a second light chain and a second heavy chain of the multispecific antibody.

In some embodiments, one of the transgenes expressed from the polycistronic expression cassette is a marker that facilitates selection of cells that have stably integrated the expression cassette into the genome and express the transgenes at high levels. For example, the selection marker may confer resistance to a cellular toxin in the cell culture medium or remedy a metabolic defect of the cell, such that the cell can survive. In some embodiments, the selection marker may be a metabolic enzyme such as glutamine synthetase (GS), dihydrofolate reductase (DHFR), or dihydroorotate dehydrogenase (DODH). In further embodiments, the host cell has a null mutation in an endogenous GS gene and the selection marker is a GS; host cells that have integrated an expression cassette comprising a GS transgene can then survive in a culture medium that lacks glutamine and/or contains methionine sulfoximine. A nonlimiting example of GS comprises SEQ ID NO:6, or an amino acid sequence at least about 85% (e.g., at least about 86, about 87, about 88, about 89, about 90, about 91, about 92, about 93, about 94, about 95, about 96, about 97, about 98, or about 99%) identical thereto. In certain embodiments, the GS transgene comprises SEQ ID NO:5, or a nucleotide sequence that is at least about 85% (e.g., at least about 86, about 87, about 88, about 89, about 90, about 91, about 92, about 93, about 94, about 95, about 96, about 97, about 98, or about 99%) identical thereto. In some embodiments, the GS is a hamster GS and may comprise SEQ ID NO:6. In other embodiments, the GS is a human GS and may comprise SEQ ID NO: 38.

In some embodiments, one of the transgenes expressed from the polycistronic expression cassette is a detectable marker that can be used to detect and isolate cells that have stably integrated the expression cassette and express the transgenes at high levels. For example, the detectable marker is a cell surface protein and cells that express this detectable marker can be detected and isolated by flow cytometry. In some embodiments, the detectable marker is a relatively small cell surface protein (e.g., a protein with fewer than 50 amino acids) such that its production does not negatively impact the titer of the target recombinant protein to be produced (e.g., a therapeutic protein such as an antibody). In further embodiments, the detectable marker is a CD52 or a variant thereof recognizable by an anti-CD52 antibody. A nonlimiting example of a CD52 variant comprises SEQ ID NO:8, or an amino acid sequence at least about 80%, (e.g., at least about 81, about 82, about 83, about 84, about 85, about 86, about 87, about 88, about 89, about 90, about 91, about 92, about 93, about 94, about 95, about 96, about 97, about 98, or about 99%) identical thereto. In certain embodiments, the CD52 transgene comprises SEQ ID NO:7, or a nucleotide sequence that is at least about 85% (e.g., at least about 86, about 87, about 88, about 89, about 90, about 91, about 92, about 93, about 94, about 95, about 96, about 97, about 98, or about 99%) identical thereto.

### D. Exemplary Polycistronic Expression Cassettes

In some embodiments, the polycistronic expression cassette comprises, from 5′ to 3′, a 5′ ITR, an insulator, a first cistron comprising an antibody light chain transgene driven by a first promoter, a second cistron comprising a selection marker transgene (e.g., a GS transgene) driven by a second promoter, a third cistron comprising an antibody heavy chain transgene driven by a third promoter, an insulator, and a 3′ ITR.

In further embodiments, the 5′ and 3′ ITR comprise SEQ ID NOs:25 and 26 (or homologous sequences as described above), the insulators comprise SEQ ID NO:10 (or a homologous sequence as described above), and the GS transgene encodes SEQ ID NO:5 (or a homologous sequence as described above).

In still further embodiments, each of the first and third promoters is a CMV promoter, e.g., a CMV promoter comprising SEQ ID NO:3 (or a homologous sequence as described above), and the second promoter is an SV40 or Δ3SV40 promoter (e.g., a promoter comprising SEQ ID NO:1 or 2, or a homologous sequence as described above).

In some embodiments, the third cistron comprises a coding sequence for CD52 (e.g., a CD52 comprising SEQ ID NO:8 or a homologous sequence as described above) upstream of the heavy chain coding sequence, where the CD52 coding sequence uses an attenuated start codon (a non-ATG start codon).

The antibody expressed by the expression cassette can be an antibody that targets a cell surface molecule (e.g., a membrane receptor on an immune cell or a tumor antigen) or a soluble antigen (e.g., a cytokine, a complement factor, or the like). For example, the antibody may target complement Factor Bb, where the light chain comprises SEQ ID NO:12 (which may be encoded by SEQ ID NO:11 or a degenerate sequence thereof) and the heavy chain comprises SEQ ID NO:14 (which may be encoded by SEQ ID NO:13 or a degenerate sequence thereof). Examples of such an expression cassette are further described below in the Working Examples (vectors #14b and #17), and their configurations are shown in FIGS. 1A, 1B, 1D, and 1E. A plasmid expression construct comprising the expression cassette is illustrated in FIG. 1C.

Additional antibodies that can be expressed by the present polycistronic expression cassette are further described in the Working Example. These antibodies are an antibody against CD40L (aka. CD154), a cell surface receptor on activated T cells, and a bispecific antibody against α-synuclein and insulin-like growth factor 1 receptor (IGF1R). For example, the antibody may target CD40L, where the light chain comprises SEQ ID NO:16 (which may be encoded by SEQ ID NO:15 or a degenerate sequence thereof) and the heavy chain comprises SEQ ID NO:18 (which may be encoded by SEQ ID NO:17 or a degenerate sequence thereof). In another example, a bispecific antibody may target α-synuclein and IGF1R, where the light chains comprise SEQ ID NO:20 (which may be encoded by SEQ ID NO:19 or a degenerate sequence thereof), the first heavy chain comprises SEQ ID NO:22 (which may be encoded by SEQ ID NO:21 or a degenerate sequence thereof), and the second heavy chain comprises SEQ ID NO:24 (which may be encoded by SEQ ID NO:23 or a degenerate sequence thereof). In another example, the antibody may target IL-1R3, where the light chain comprises SEQ ID NO:40 (which may be encoded by SEQ ID NO:39 or a degenerate sequence thereof) and the heavy chain comprises SEQ ID NO:42 (which may be encoded by SEQ ID NO:41 or a degenerate sequence thereof).

### II. Recombinant Protein Expression

An expression construct (e.g., a DNA plasmid) comprising the polycistronic expression cassette herein may be used to transfect a mammalian host cell for production of the encoded proteins. A transposase that recognizes the ITRs in the cassette is transiently introduced to the same host cell, e.g., through a separate expression vector (e.g., a non-integrating expression vector or an mRNA).

The mammalian host cell may be, for example, a CHO cell, a HEK cells, a BHK cell, a 293 cell, a HeLa cell, a NS0 cell, a Sp2/0 cell, a U2OS cell, and the like. The host cells that have stably integrated the polycistronic expression cassette (e.g., the transposon unit) into the genome may be selected based on the selection marker (e.g., GS or DHFR) and detectable marker (e.g., a CD52) encoded by the cassette.

In some embodiments, the mammalian host cells may integrate multiple copies of the polycistronic expression cassettes, e.g., at least about 10 copies, at least about 20 copies, at least about 30 copies, at least about 40 copies, at least about 50 copies, at least about 60 copies, at least about 70 copies, at least about 80 copies, at least about 90 copies, at least about 100 copies, at least about 110 copies, or at least about 120 copies.

In some embodiments, the mammalian host cells comprising the polycistronic expression cassette may produce the recombinant protein of interest (e.g., an antibody) at a titer of at least about 0.5 g/L, at least about 1.0 g/L, at least about 1.5 g/L, at least about 2.0 g/L, at least about 2.5 g/L, at least about 3.0 g/L, at least about 3.5 g/L, at least about 4.0 g/L, at least about 4.5 g/L, at least about 5.0 g/L, at least about 5.5 g/L, at least about 6.0 g/L, at least about 6.5 g/L, at least about 7.0 g/L, at least about 7.5 g/L, at least about 8.0 g/L, at least about 8.5 g/L, at least about 9.0 g/L, or at least about 9.5 g/L.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety, as if each individual reference were specifically and individually indicated to be incorporated by reference in its entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

According to the present disclosure, back-references in the dependent claims are meant as short-hand writing for a direct and unambiguous disclosure of each and every combination of claims that is indicated by the back-reference. Further, headers herein are created for ease of organization and are not intended to limit the scope of the claimed invention in any manner.

In order that this invention may be better understood, the following examples are set forth. These examples are for purposes of illustration only and are not to be construed as limiting the scope of the invention in any manner.

### EXAMPLES

The materials and methods used in the Examples are first described below.

### Construction of DNA Expression Vectors

Each evaluated sequence was inserted into the piggyBac® base vector (14.7.1) via either restriction enzyme cloning or Gibson Assembly (New England Biolabs) when restriction enzyme sites were not available. For restriction enzyme cloning, NheI and Pac sites were utilized for insertion into the base vector. Gibson Assembly using 4-7 inserts and 15-20 base pair insert overlapping was utilized to generate the more complex double gene vector topology re-arrangement sequences. All final plasmid maps were sequence-verified by GENEWIZ (Azenta Life Sciences). The transposase used in these experiments had a sequence of SEQ ID NO:37.

### CHO Cell Line and Cell Culture

An internally generated GS-knockout CHO-K1 cell line (16K55), derived from European Collection of Authenticated Cell Cultures (ECCAC), was used in these studies. Cells were grown and maintained in a controlled shaking incubator (Kuhner) at 37 °C, 5% CO2 and 80% humidity, with shaking speeds of 125 rpm (shake flasks) or 260 rpm (spin tubes). The parental cell line was grown in CD CHO medium (Life Technologies) supplemented with 8 mM L-glutamine and 50 mg/L dextran sulfate prior to selection. Pool transfections were performed via electroporation using a Gene Pulser Xcell (BioRad Laboratories) in CD DG44 medium (Life Technologies) supplemented with 8 mM L-glutamine and 0.18% Pluronic F-68. All piggyBac® plasmid DNA was transfected as sterile circular DNA with either plasmid DNA or an RNA encoding a transposase. During selection, the CHO cells were cultured in CD CHO medium supplemented with 50 mg/L dextran sulfate and various levels of methionine sulfoximine (MSX) for stable pool generation. Selecting pools were considered finished after achieving pool viabilities >95% and doubling times <32 hours.

### FLARE Reporter System

A FLARE (FLow cytometric Attenuated Reporter Expression) system was used for the rapid detection and selection of pools or clones that produced high levels of recombinant proteins. In this system, a sequence-altered human CD52 cell surface reporter gene with attenuated expression is co-expressed with the protein biologic of interest. The reporter gene, utilizing a non-AUG start codon, is positioned immediately upstream of the gene encoding the protein biologic, which uses the standard AUG start codon. This arrangement results in low expression of the reporter gene and high expression of the protein biologic. Both genes are transcribed from a single mRNA molecule but are translated independently. In a standard monoclonal antibody expression format, the reporter gene is co-expressed with the heavy chain sequence. This design ensures that the expression of the reporter gene on the cell surface correlates with the level of protein biologic expression, making the reporter gene a predictive indicator of the protein biologic's expression. This approach enables efficient identification and selection of high-producing clones in the development of recombinant biologics. Vectors #10, #13, #14, #14b, #14c, #14d, and #20 contain the CD52 FLARE reporter.

### Flow Cytometry Analysis of Reporter Expression

After pool selection was completed, reporter expression was evaluated where cells were incubated with FITC-conjugated anti-human CD52 antibody for 30 min at 4 °C, with cold CD-CHO medium washes before and after antibody incubation. For reporter analysis, cells were resuspended in cold CD-CHO medium at approximately 2×10⁶ viable cells/mL and acquired on the FACSCelesta™ flow cytometer (BD Biosciences). Data analysis was performed using the FlowJo™ software program. Flow cytometry results are displayed as a measure of the geometric mean fluorescence intensity (GMFI) and quantified in relative fluorescence units (RFU).

### Reporter Bulk Enrichment and Clone Generation, Screening and Expansion

After pool selection was completed, 20-80×10⁶ cells were incubated with FITC-conjugated anti-human CD52 antibody for 30 min at 4 °C, with cold CD-CHO medium washes before and after antibody incubation. These cells were resuspended in cold CD CHO/DMEM:F12 medium (Life Technologies) at a concentration of 2×10⁷ viable cells/mL and sorted for bulk enrichment on the BDInflux™ flow cytometer (BD Biosciences) using an argon laser emitting at 488 nm and detecting FITC emission with a 530/30 filter. Selection of cells was based on viable cell scatter plots (FSC-A and SSC-A) with doublet discrimination and cells exhibiting the highest FITC fluorescence. Cells were then sorted at the top 5-10% FITC expression as a bulk collection, plated into a 6-well static plate and allowed to recover for 24 hours in CD CHO medium supplemented with 50 mg/L dextran sulfate and MSX (if applicable for selection purposes).

After 24 hours of recovery, the bulk pool was prepared for clone generation using the Beacon® Optofluidic System (Bruker Cellular Analysis). The cells were counted and resuspended in EX-CELL® CHO Cloning medium (Sigma-Aldrich) supplemented with InstiGRO™ CHO (Advanced Instruments) at a cell concentration of 4×10⁶ viable cells/mL. Cells were then single-cell imported onto the Beacon into OptoSelect® 1750 chips for clone generation. On day 3-4 post-import and after clones were allowed to grow to 6-30 total cells in each NanoPen® chamber, all clones were screened using a fluorescent diffusion gradient assay for secreted protein biologic targets. The top expressing clones were then exported into each well of a static 96-well plate. Twice per week, clones were scanned using the CellMetric® (Advanced Instruments) for well confluence. Once a well achieved >45% confluence, clones were FACS-screened for the reporter as described above after pool selection. Utilizing the reporter expression, the top clones were then expanded from 96-well plates to 24-well static plates, 24-deep well plates, and ultimately to spin tubes in CD CHO medium supplemented with 50 mg/L dextran sulfate and MSX (if applicable for selection purposes). Clones were considered finished with expansion after achieving clone viabilities in spin tubes >98% and doubling times <30 hours.

### Analysis of Pool and Clone Protein Production

After completion of pool selection or clone expansion, cells were inoculated into shake tubes at 5×10⁵ viable cells/mL for batch culture analysis with no feeding. On day 7 of batch culture, conditioned medium was harvested, clarified by centrifugation and analyzed for titer by Octet® Protein A methods.

Pools and clones were also analyzed on the Ambr®15 (Sartorius) for fed-batch analysis. The day before inoculation (day −1), Ambr®15 reactors were prepared by adding 11 mL of in-house medium and 20 μL anti-foam solution (20% diluted in WFI) to each reactor. Each pool or clone was inoculated as follows: An aliquot of the seed train culture was counted on the Vi-CELL™ XR (Beckman Coulter) and, based on the cell count result, 30×10⁶ viable cells were harvested from the culture and transferred into a 15-mL tube. The tube was centrifuged, the supernatant was removed, and the cells were resuspended in 4 mL of in-house medium that had been pre-equilibrated to 37 °C. An Ambr®15 reactor was inoculated with the 4 mL cell suspension to achieve a target seed density of 2×10⁶ viable cells/mL in a total working volume of 15 mL. Daily feeds were initiated on day 1 with feed rates of 0.0012 mL/Mvc (million viable cells) and 0.00008 mL/Mvc for feed 1 and feed 2, respectively. From day 3 onward, 200 μL of culture was sampled daily for IgG and lactate dehydrogenase (LDH) measurements on the Cedex® Bio HT (Roche). 450 μL of culture was sampled daily for cell counts, viability, cell size, glucose, lactate, glutamine, glutamate, ammonium, potassium, calcium, sodium, pH, pCO₂, pO₂, and osmolality on a BioProfile® FLEX2 (Nova Biomedical). Additionally, on days 7 and 10 (or 12), conditioned medium was harvested, clarified by centrifugation and analyzed for titer by Octet® Protein A methods.

### Transcript Level Analysis

To measure the level of transcript, cells were harvested through centrifugation after completion of selection. RNA was extracted using Qiagen AllPrep DNA/RNA kit based on the manufacturer's protocol. RNA samples were subjected to reverse transcription to synthesize cDNA using SuperScript™ IV Reverse Transcriptase kit (ThermoFisher, cat #18090010). The QX600 Droplet Digital™ PCR System (Bio-Rad Laboratories) was used to quantify the heavy chain and light chain in a duplex reaction with GAPDH as a reference. ddPCR supermix for probes (no dUTP) (Bio-Rad Laboratories, cat #1663025) was used based on the manufacturer's protocol. Primers and probes, including fluorescence dyes and quenchers, were designed based on parameters suited for ddPCR (Table 1). Droplets were generated using the AutoDG Droplet generator (Bio-Rad Laboratories). PCR reactions were performed on a C1000 Touch™ Thermal Cycler (Bio-Rad Laboratories) using the following program: 1 cycle at 95 °C for 10 min, 40 cycles at 94 °C for 30 s and at 58 °C for 1 min, 1 cycle at 98 °C for 10 min and hold at 4 °C. After PCR amplification, droplets were measured in the QX200 ddPCR Droplet Reader, and relative transcript levels were analyzed using QX manager software (standard edition, Bio-Rad Laboratories).

TABLE 1
Primers Used for ddPCR

| Sequence name | Sequence | SEQ ID NO |
|---|---|---|
| Heavy chain forward primer | 5′-CTCCAAAGCCTAAGGACACC-3′ | 27 |
| Heavy chain reverse primer | 5′-CGTACCAATTGAACTGCACC-3′ | 28 |
| Heavy chain probe | 5′-CGGACCCCTGAAGTGACCTGCGTGG-3′ | 29 |
| Light chain forward primer | 5′-CCCAAGAGTCTGTGACCGA-3′ | 30 |
| Light chain reverse primer | 5′-GTACACCTTGTGCTTCTCGT-3′ | 31 |
| Light chain probe | 5′-CCACACTGACCCTGTCCAAGGCCGA-3′ | 32 |
| GAPDH Forward Primer | 5′-TCAAGAAGGTGGTGAAGCAG-3′ | 33 |
| GAPDH Reverse Primer | 5′-CAAAGGTGGAAGAGTGGGAG-3′ | 34 |
| GAPDH Probe | 5′-TGGGCTACACCGAGGACCAGGTTGT-3′ | 35 |

### Example 1: Design of DNA Expression Vectors

Expression vectors were constructed with a human CMV promoter (SEQ ID NO:3) driving expression of a coding sequence for the light chain (LC) gene of an anti-Factor Bb antibody (SEQ ID NO: 11) and either a human CMV (SEQ ID NO:3) or hamster β-actin promoter (SEQ ID NO:4) driving expression of a coding sequence for the heavy chain (HC) gene sequence of the anti-Bb antibody (SEQ ID NO:13) (FIGS. 1A-1B). In most vector configurations, a truncated version of SV40 (Δ3) (SEQ ID NO:2) was placed upstream of a glutamine synthetase (GS) gene (SEQ ID NO:5) to aid in attenuating GS expression and facilitate pool expression. In several constructs, an altered human CD52 reporter gene (SEQ ID NO:7) was inserted immediately upstream of the HC gene to enable pool screening and clone production. For vectors #13-16, the expression cassette configuration followed the pattern 5′-GS-LC-HC-3′, with the exceptions of sequence #14b (5′-LC-GS-HC-3′) and sequence #14c (5′-GS-HC-LC-3′). For vectors #17-22, the expression cassette topology varied between the three positions to help identify the most productive vector arrangement for this study.

### Example 2: Stable Pool Expression Analysis and Batch Productivity

A comprehensive stable pool expression analysis was performed on the experimental vectors, investigating batch and fed-batch pool productivity. Pool generation was conducted in duplicate for each vector configuration, with a human CMV promoter controlling the light chain gene expression in all cases. For vectors #13 and #14 (with reporter) and #15 and #16 (without reporter), either a human CMV or hamster β-actin promoter was used to drive the heavy chain gene expression. Vectors #18, #19, #20, and #21 incorporated the heavy chain gene in position 1, while vectors #19, #20, #21, and #22 featured the GS in position 3 (FIGS. 1A-1B). In addition to these topological variations, vectors #21 and #22 introduced further modifications, including the integration of the ubiquitous chromatin opening element (A2UCOE) in vector #21, and the incorporation of a full-length SV40 promoter (SEQ ID NO:1) upstream of the GS gene in vector #22. Vector #17's configuration is identical to vector #14b, except for the absence of the reporter (CD52) sequence.

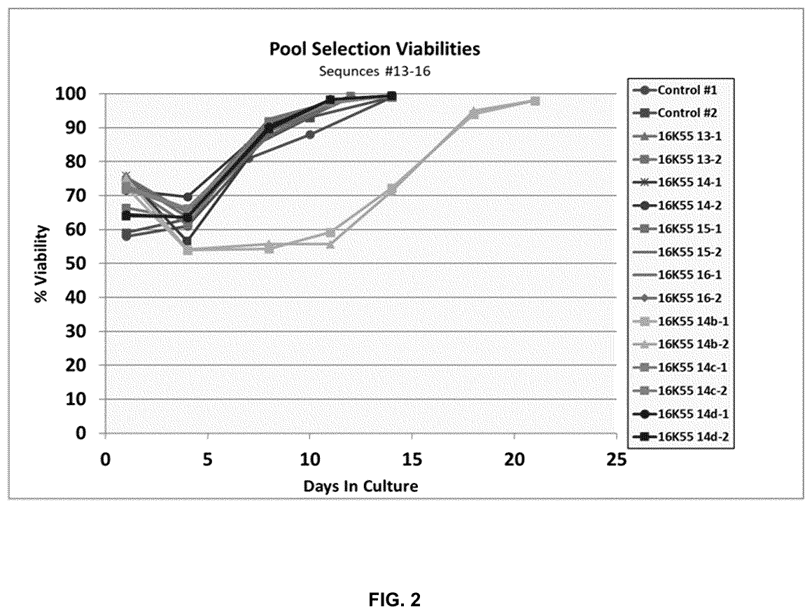

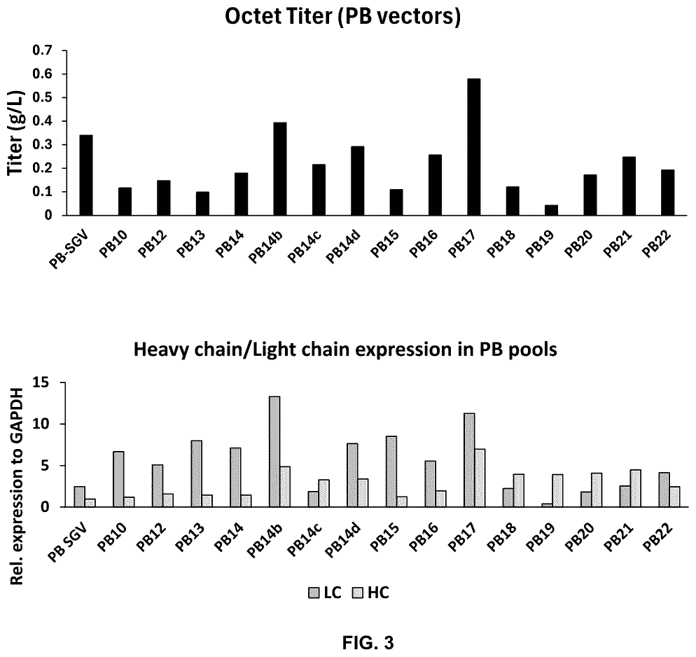

Pool viability profiles were similar during pool selection (FIG. 2). However, day 7 batch pool productivity was higher when the human CMV promoter controlled the HC gene. Specifically, vector #14 (with the reporter) exhibited higher pool titers than vector #13, and vector #16 (without the reporter) showed higher titers than vector #15 (FIG. 3). These results indicate that the double human CMV promoters driving both the LC and HC genes were optimal for this vector orientation (5′-GS-LC-HC-3′ configuration). Interestingly, the vectors without the reporter (#15 and #16) demonstrated higher pool productivity than those with the reporter gene (#13 and #14).

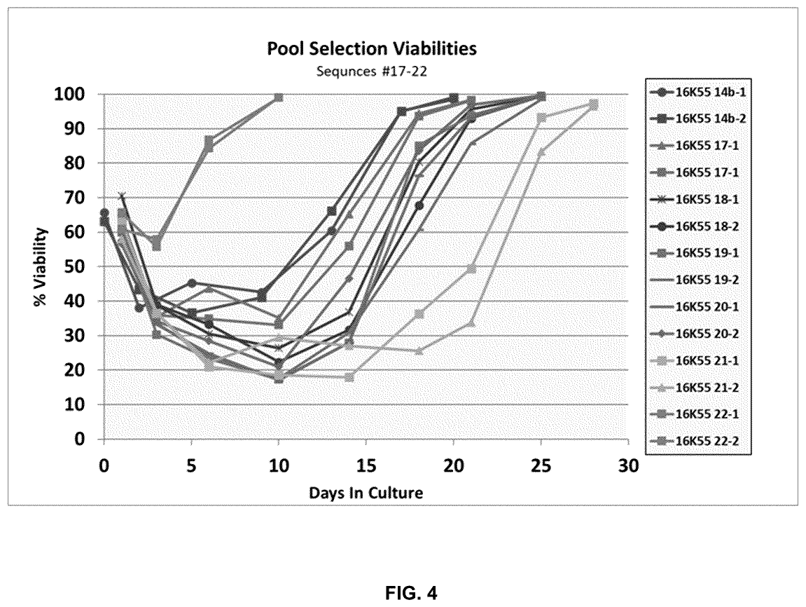

In vectors #17-22, pool viability profiles exhibited variation across different sequences during pool selection, primarily due to the re-positioning of the GS gene within the variable vector gene cassettes (FIG. 4). Pool selection concluded rapidly for vector #22 within 10 days, likely owing to the inclusion of the full-length SV40 promoter upstream of the GS gene. Pool selections for other vectors completed within 25 days, except that pool selection was not completed for vector #21 until day 28. Notably, the two vectors exhibiting the highest pool productivity were #14b and #17 (FIG. 3). Both vectors shared the 5′-LC-GS-HC-3′ configuration, but vector #14b included the reporter sequence while vector #17 did not. Vectors with other configurations displayed significantly lower pool titers using a 7-day batch assay, with values consistently at or below 200 mg/L.

Further evaluation of vector #14 derivatives (#14b, #14c, and #14d) was conducted. Vector #14b has a 5′-LC-GS-HC-3′ configuration. Vector #14c has a 5′-GS-HC-LC-3′ configuration. Vector #14d retains the 5′-GS-LC-HC-3′ configuration but incorporates a ubiquitous chromatin opening element (UCOE) upstream of the human CMV promoter driving the HC gene. During pool selection, vector #14b exhibited increased pool stringency (FIG. 2) and achieved the highest day 7 batch pool titers after completion of selection (FIG. 3). To compare vector performance, all piggyBac®/double-gene vector pools were compared with control random integration/single-gene vector (SGV) pools, where co-transfection of the monoclonal antibody heavy and light chain plasmids was performed and the hamster β-actin promoter was used to drive both heavy and light chain gene expression. The batch pool titer results indicated that the piggyBac®/double-gene vector #14b generated comparable pool productivity to the control co-transfected random integration/single-gene vector pools (FIG. 3).

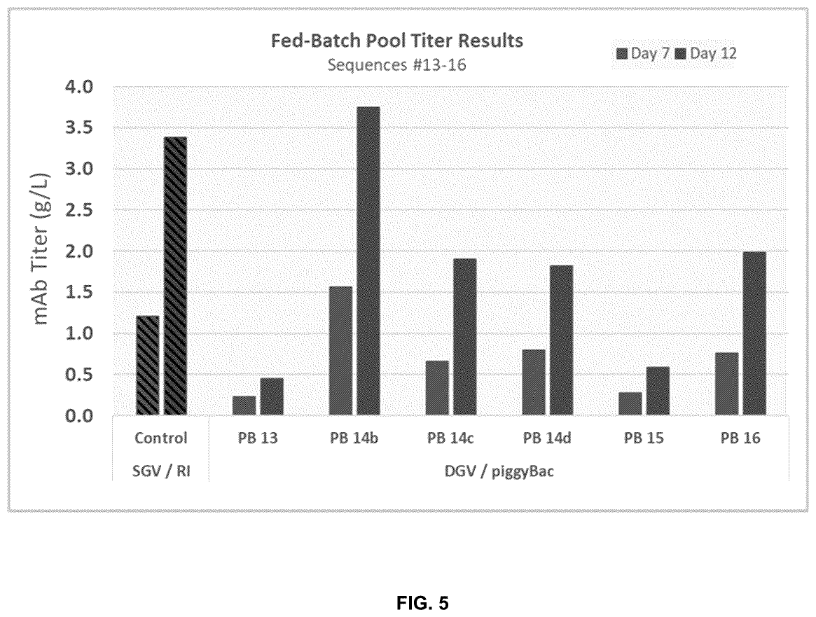

Fed-batch pool productivity was also assessed for piggyBac®/double-gene vectors #13-16 as well as vectors #14b, #14c and #14d. Fed-batch analysis was performed using the Ambr® 15 system, with conditioned medium harvested on days 7 and 12 for productivity analysis (see Methods & Materials). Under fed-batch conditions, the results show that vector #14b exhibited the highest fed-batch production among the piggyBac®/double-gene vector configurations, also slightly outperforming the random integration/single-gene vector control pools (FIG. 5). These findings suggest that the 5′-LC-GS-HC-3′ vector configuration (#14b) is optimal for pool productivity among vectors with different configuration (vectors #13-16).

### Example 3: RNA Transcript Levels Analysis

RNA transcript levels for the heavy and light chains of anti-Factor Bb antibody were analyzed to better understand the relationship between RNA transcript levels and overall pool productivity (FIG. 3). Identifying a correlative relationship between the two would help elucidate the impact and benefits of various vector configurations and facilitate the identification of the optimal double-gene vector configuration for protein production.

Among vector #14 derivatives, #14b exhibited increased heavy and light chain RNA transcript levels, showing 3-fold and 1.8-fold increases, respectively, compared to the original vector #14 (FIG. 3). This increase was attributed to the repositioning of the GS gene to position 2 and the light chain to position 1. Vector #14c showed a 2.5-fold decrease in light chain expression compared to vector #14 when the light chain was moved from position 2 to 3. Remarkably, vector #14d demonstrated increases in both heavy (3.5-fold) and light chain (1.6-fold) RNA transcript levels compared to vector #14, despite only the insertion of a UCOE sequence upstream of the heavy chain promoter.

RNA transcript levels for the heavy and light chains of the anti-Factor Bb antibody for vectors #17-22 were also analyzed (FIG. 3). The results reveal significantly higher heavy chain transcript levels in vector configurations where the heavy chain gene sequence occupies position 1 (vectors #18, #19, #20, and #21). Similarly, light chain transcript levels were notably higher in vector configurations with the light chain gene sequence in position 1 (vectors #14b, #17, and #22). Interestingly, vector #19 exhibited the lowest light chain transcript levels despite having the light chain in position 2, whereas vector #18 demonstrated robust light chain transcript levels despite placing the light chain in position 3. The re-positioning of the GS gene (position 3 and position 2, respectively) between these two vector configurations and the additional selective pressure may have played a role in the difference in light chain expression.

In summary, antibody titer was the highest for pools expressing antibodies derived from the #14b or #17 configuration. These two vectors also had the highest heavy chain transcript level while maintaining an excess of light chain transcript (excess light chain is important for proper folding and final titer). Overall, significant differences in RNA transcript levels were observed with various vector topology changes, revealing a general cassette expression hierarchy of position 1>position 3>position 2. However, there were also examples where changes in RNA transcript levels occurred despite unchanged cassette positions, such as the light chain transcript levels between sequences #14 and #14d.

### Example 4: CD52 Reporter Expression Analysis

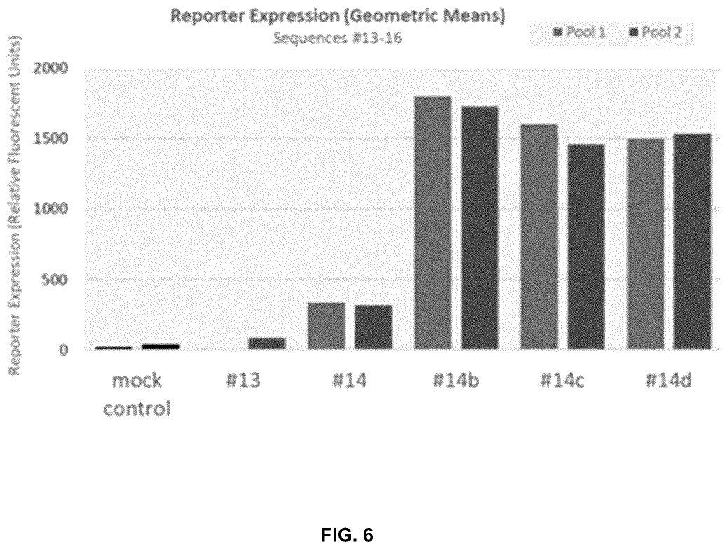

CD52 reporter expression was analyzed for vectors #13-16, which included those containing the reporter gene sequence (vectors #13, #14, #14b, #14c and #14d). The reporter gene was positioned immediately upstream of the heavy chain gene sequence, ensuring a tightly linked correlation between reporter and heavy chain expression. This close coupling resulted in reporter expression variations correlating with both pool and clone expression for monoclonal antibody formats. Vector #14b exhibited the highest reporter expression among all vector configurations (FIG. 6). Vectors #14c and #14d also demonstrated higher reporter expression compared to parental vector #14. Notably, vector #14d, which retains the 5′-GS-LC-HC-3′ configuration but includes a UCOE sequence upstream of the heavy chain, showed a 4-fold increase in reporter expression compared to vector #14 without the UCOE sequence.

### Example 5: Clone Expression Analysis

Following the pool productivity screens, it was determined that vectors #14b and #17, both containing the 5′-LC-GC-HC-3′ vector configuration (with vector #17 lacking the reporter gene), generated the highest pool productivity and optimal HC/LC RNA transcript ratio results. To assess whether clones derived from this vector configuration were also productive, clones were generated using vector #14b to evaluate the productivity gain from the resulting clones. Vector #14b was selected for clone generation due to the presence of the reporter sequence, which enabled bulk sorting prior to cloning.

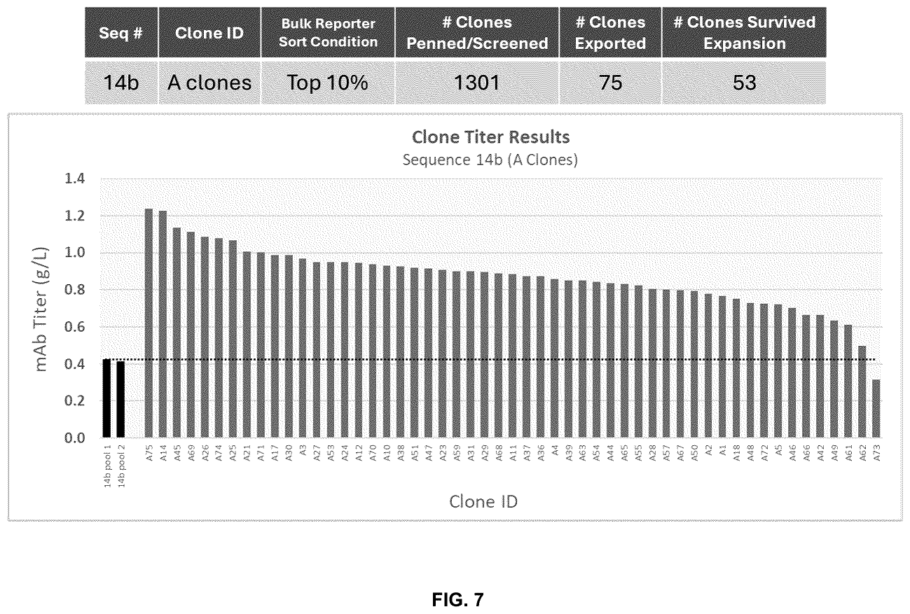

Two vector #14b pools were combined and bulk sorted via the reporter to enrich for the top 10% of cells. The enriched cells were plated into 6-well plates and allowed to recover overnight. The following day, the bulk-enriched pool was loaded onto the Beacon® Optofluidic System, and single cells were imported onto a single Beacon® OptoSelect 1750 chip for clone generation. 1301 clones grew within pens and were screened for the secreted protein biologic target (FIG. 7). The top 75 clones (designated as A clones) were selected for export into 96-well plates. These clones were subsequently expanded through a series of culture stages, including 96-well plates, 24-well plates, 24-deep well plates, and spin tube via our cell line development procedure.

Out of the 75 successfully exported clones, 53 (71%) survived the spin tube expansion stage and were evaluated for batch productivity. The top clone in the day 7 clone batch titer evaluations achieved 1.22 g/L (FIG. 7). The original #14b presort pools are also depicted for comparison, showing that the majority of resulting clones exhibited significant titer gains relative to the presort pool productivities.

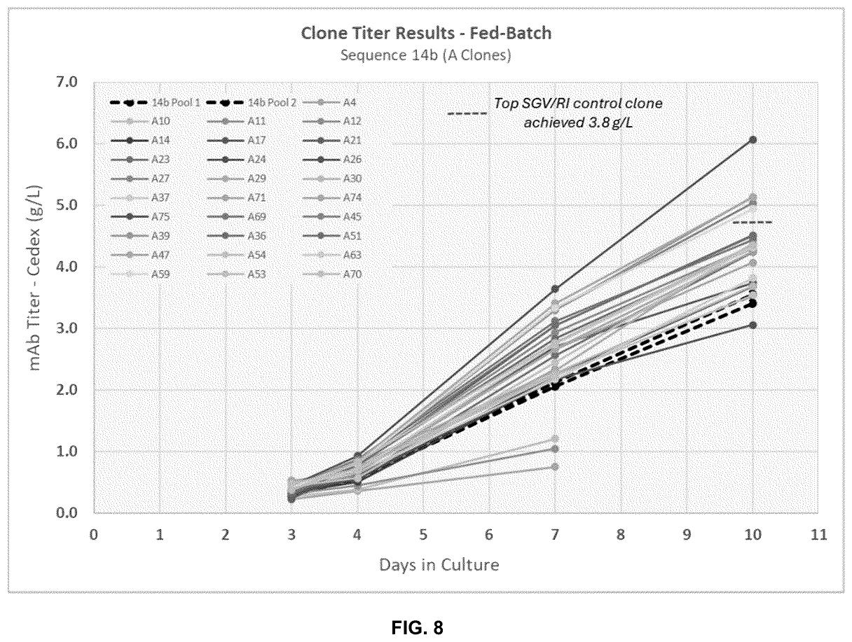

The top resulting clones were further evaluated in a fed-batch productivity environment. The top 28 clones from the batch titer analysis were thawed and inoculated in an Ambr® 15 10-day fed-batch process to assess peak titer productivities using a fed-batch methodology. The Ambr® 15 fed-batch system is known to better replicate downstream bioreactor fed-batch conditions during cell line development programs. The top clone of the fed-batch clone titer productivities resulting from vector #14b pools achieved a peak titer of 6.1 g/L on day 10, with most clones demonstrating higher fed-batch titer productivities compared to the presort pools (FIG. 8, dotted black line). For comparison, the top clone from the random integration/single-gene vector co-transfection control, which had been previously tested for this molecule, achieved a significantly lower titer of 3.8 g/L on day 10 in an Ambr® 15 fed-batch process. These results demonstrate that the piggyBac®/double-gene vector strategy successfully produced a higher-productive clone compared to the traditional random integration/single-gene vector transfection approach.

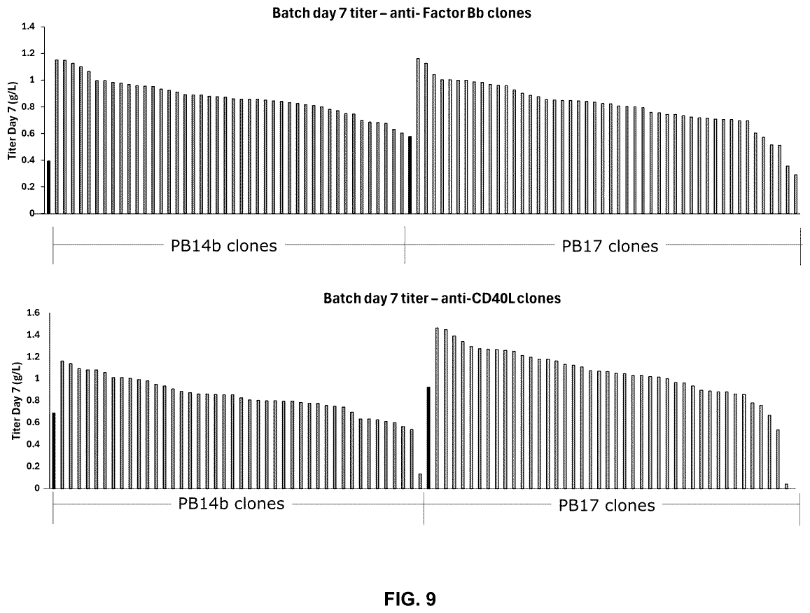

Clone expression analysis was also conducted for vector #17, as above. The top clones in the day 7 terminal batch culture achieved similar titers to those of vector #14b (FIG. 9).

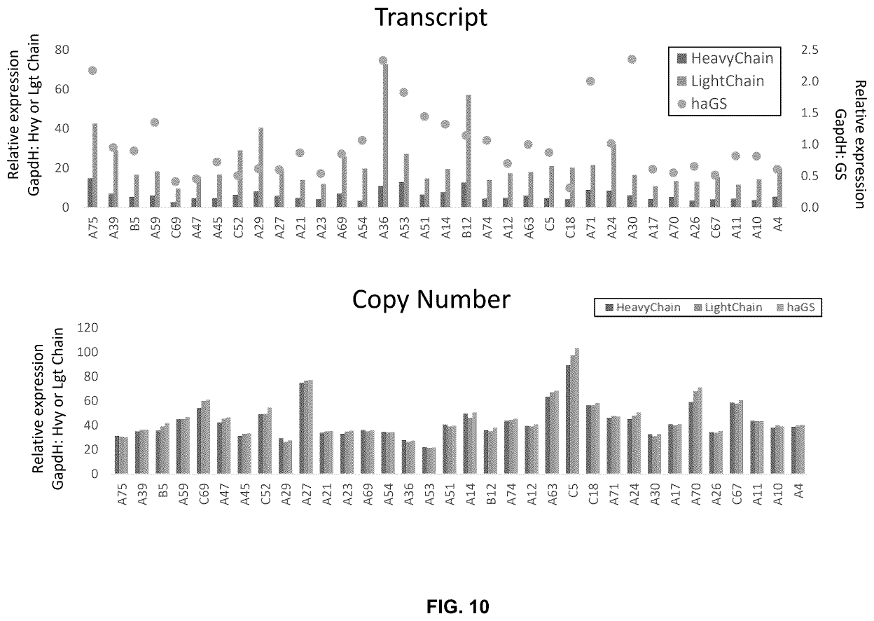

Further, the vector #14b clones were evaluated for the heavy chain, light chain, and hamster GS transcript levels (FIG. 10). The relative expression of the anti-Factor Bb heavy and light chains was calculated relative to GAPDH. Most clones yielded an HC/LC transcript ratio between 1:2 and 1:4. The GS transcript expression was much lower than that of the HC and LC transcripts (2 or less). This is consistent with lower expression from position 2 in the vector topology and expression under the attenuated Δ3SV40 promoter. Though the various clones yielded favorable transcript levels, this did not correlate to the antibody expression titers (FIG. 9).

The copy number of HC, LC, and glutamine synthetase elements were also analyzed for each of the vector #14b clones (FIG. 10). For most of the clones, the copy number ranged between 20 and 70 copies. The copy number of each of the components also appeared to be similar across each clone, consistent with expression from the same vector.

### Example 6: Batch Titer Analysis of Additional Antibody Candidates

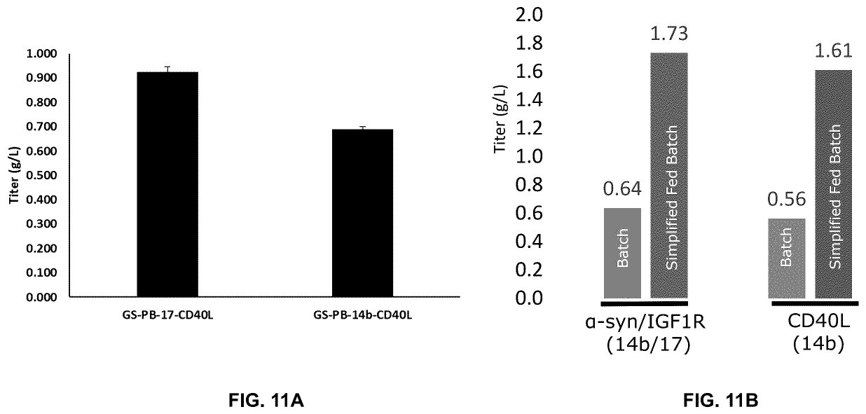

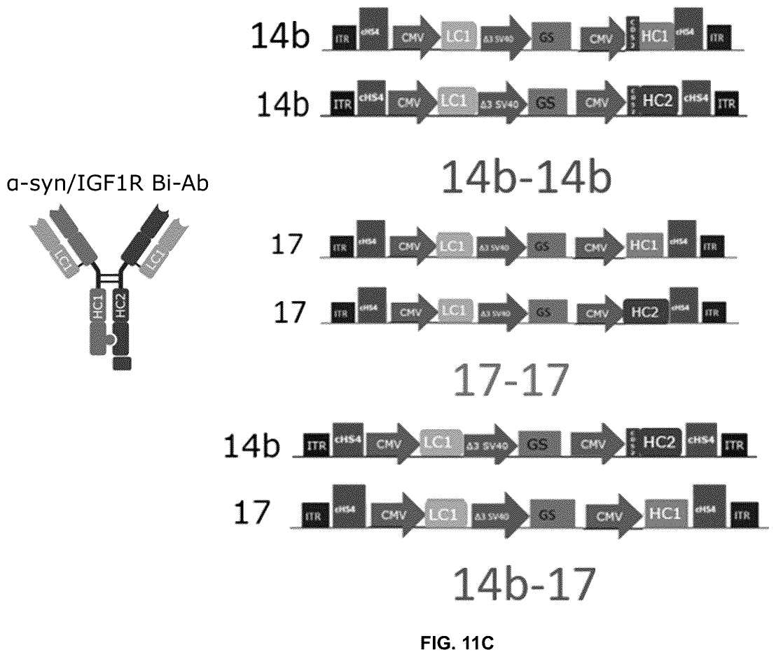

To confirm that the high titers achieved by vectors #14b and #17 were not specific to the anti-Factor Bb antibody production, the heavy and light chain sequences of another antibody, anti-CD40L (SEQ ID NOs:16 and 18), were inserted into each vector. Titers from day 7 of a batch culture of pools expressing the anti-CD40L antibody in either vector were analyzed (FIG. 11A). Both vectors exhibited high titers of the anti-CD40L antibody, indicating sufficient expression of the antibody, as was observed for the anti-Factor Bb antibody.

Further, vector #14b expressing anti-CD40L was compared to the bispecific anti-α-synuclein/anti-IGF1R antibody (SEQ ID NOs:20 and 22) expressed from vectors #14b and #17. Cells were transfected with a 50% mixture of the two vectors comprising ABL 301: vector #14b expressing a light chain, a GS selectable marker, and a heavy chain fused to a scFv, and vector #17 expressing a second light chain, a GS selectable marker, and a heavy chain. The antibody titers were analyzed after batch and simplified fed batch expression (FIG. 11B). In both experiments, the simplified fed batch expression yielded higher antibody titers. Further, the antibody titers of the bispecific antibody (bi-ab) were comparable to that of anti-CD40L, indicating that these vector topologies can adequately express various antibodies.

### Example 7: Batch Titer Analysis of an Anti-IL-1R3 Antibody

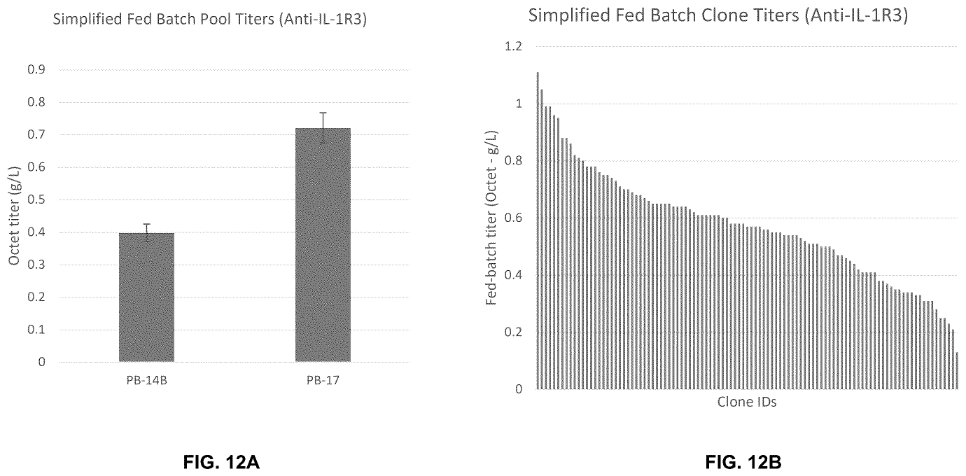

To further assess the productivity of vectors #14b and #17, the heavy chain sequence (SEQ ID NO: 42) and light chain sequence (SEQ ID NO: 40) of a monospecific anti-IL-1R3 antibody, were inserted into each vector. Titers from day 7 of a batch culture of pools expressing the anti-IL-1R3 antibody in either vector were analyzed and the results are shown in FIG. 12A. Vector #17, which lacks the FLARE reporter, demonstrated higher titers of the anti-IL-1R3 antibody than vector #14b.

To assess the productivity of anti-IL-1R3 production from vector #17, clones were generated using vector #17 to evaluate the productivity gain from the resulting clones.

Vector #17 clones were enriched, screened, expanded, and evaluated for batch productivity and the results are shown in FIG. 12B. Clones expressing anti-IL-1R3 from vector #17 showed robust titers reaching over 1 g/L for top clones.

### SEQUENCES

The table below shows the sequences described herein (SEQ: SEQ ID NO).

| SEQ | Description | Sequence |
|---|---|---|
| 1 | SV40 | gctgtggaatgtgtgtcagttagggtgtggaaagtccccaggctccccag |
| | Promoter | caggcagaagtatgcaaagcatgcatctcaattagtcagcaaccaggtgt |
| | | ggaaagtccccaggctccccagcaggcagaagtatgcaaagcatgcatct |
| | | caattagtcagcaaccatagtcccgcccctaactccgcccatcccgcccc |
| | | taactccgcccagttccgcccattctccgccccatggctgactaattttt |
| | | tttatttatgcagaggccgaggccgcctcggcctctgagctattccagaa |
| | | gtagtgaggaggcttttttggaggcctaggcttttgcaaa |
| 2 | Δ3SV40 | ctgtggaatgtgtgtcagttagtcccgcccctaactccgcccagttccgc |
| | Promoter | ccattctccgccccatggctgactaattttttttatttatgcagaggccg |
| | | aggccgcctcggcctctgagctattccagaagtagtgaggaggctttttt |
| | | ggaggcctaggcttttgcaaa |
| 3 | CMV | cgccatgttgacattgattattgactagttattaatagtaatcaattacg |
| | Promoter | gggtcattagttcatagcccatatatggagttccgcgttacataacttac |
| | | ggtaaatggcccgcctggctgaccgcccaacgacccccgcccattgacgt |
| | | caataatgacgtatgttcccatagtaacgccaatagggactttccattga |
| | | cgtcaatgggtggagtatttacggtaaactgcccacttggcagtacatca |
| | | agtgtatcatatgccaagtacgccccctattgacgtcaatgacggtaaat |
| | | ggcccgcctggcattatgcccagtacatgaccttatgggactttcctact |
| | | tggcagtacatctacgtattagtcatcgctattaccatggtgatgcggtt |
| | | ttggcagtacatcaatgggcgtggatagcggtttgactcacggggatttc |
| | | caagtctccaccccattgacgtcaatgggagtttgttttggcaccaaaat |
| | | caacgggactttccaaaatgtcgtaacaactccgccccattgacgcaaat |
| | | gggcggtaggcgtgtacggtgggaggtctatataagcagagctcgtttag |
| | | tgaaccgtcagatcgcctggagacgccatccacgctgttttgacctccat |
| | | agaagacaccgggaccgatccagcctccgcggccgggaacggtgcattgg |
| | | aacgcggattccccgtgccaagagtgacgtaagtaccgcctatagagtct |
| | | ataggcccacccccttggcttcttatgcatgctatactgtttttggcttg |
| | | gggtctatacacccccgcttcctcatgttataggtgatggtatagcttag |
| | | cctataggtgtgggttattgaccattattgaccactcccctattggtgac |
| | | gatactttccattactaatccataacattgcagaatcaccaccgagagga |
| | | taatgatcaaggagtacggctctttgccacaactctctttattggctata |
| | | tgccaatacactgtccttcagagactgacacggactctgtatttttacag |
| | | gatggggtctcatttattatttacaaattcacatatacaacaccaccgtc |
| | | cccagtgcccgcagtttttattaaacataacgtgggatctccacgcgaat |
| | | ctcgggtacgtgttccggacatgggctcttctccggtagcggcggagctt |
| | | ctacatccgagccctgctcccatgcctccagcgactcatggtcgctgtaa |
| | | ggccgtgacaacatacacgaggattggataggcctcggcagctccttgct |
| | | cctaacagtggaggccagacttaggcacagcacgatgcccaccaccacca |
| | | gtgtgccgcacaaggccgtggcggtagggtatgtgtctgaaaatgagctc |
| | | ggggagcgggcttgcaccgctgacgcatttggaagacttaaggcagcggc |
| | | agaagaagatgcaggcagctgagttgttgtgttctgataagagtcagagg |
| | | taactcccgttgcggtgctgttaacggtggagggcagtgtagtctgagca |
| | | gtactcgttgctgccgcgcgcgccaccagacataatagctgacagactaa |
| | | cagactgttcctttccatgggtcttttctgcagtcaccgtccttgacacg |
| 4 | Hamster β- | agtcctcattgacaggcagtattcgctctgccccacccccacctgtgaat |
| | actin | tgcagggctggcaggtcctcaggcagctggcaaaccgcctgaacaactga |
| | Promoter | gagatacagggccagggccagggcagtcccgtcccccggaggcagggagg |
| | | ggacgtgctgggaaagttctctctctcaggcccaggttggtgactgcaga |
| | | aggcttctgtcaaatctcttttgtgggaaccacagagtagccctgaacgt |
| | | gggggtgtgcttccagtatactctggggtcaccctttccatactggaggc |
| | | ctctgcaacttcaaaatgctctgctaccaacctagcacaaggaagttggt |
| | | ccagcctccccacgcagggccactgctgcagtccatatatggactaagcc |
| | | ttccttggtttcaacacctacactcactgagcccctactatgtgtatgca |
| | | gagccgagacaggccctgagcatctcatctgaagcacccttcttgcctaa |
| | | attcagttttctgtcactttctcccaggaggtgtgtgtccctctaagcta |
| | | agccaggggtccctcacccctgccccactcccatccctagtgtaggtatc |
| | | agctgaagagcttcctgagcagaacactcttgggtgctgacattttgata |
| | | aataggcccatgtttaggagagcaggggtccgggggcgggagatcttctc |
| | | tggtggattgagggctccaagaactactctttgagcacgctgcccctccc |
| | | agagtccccacagcctccagatggactagaacacagttcggctgtggctg |
| | | cacataactaacagaggatagatggtgggtcccagcccaacagtgcctgg |
| | | caatcacccagagccaccagctaacggccttggcttagttttttgcctgg |
| | | gtgtgatcaggcagccctccaaaactgcccggactccatgacaagttttg |
| | | cttgttctatagagcacagttcctttctaggtctggggcaagggacatcg |
| | | ggagacatcttcctgcaacagctccagtcactggaccaccaggctcgccc |
| | | tgtctttggtgtgtggccctgagtctcctaagtggcccaaacctgtgaag |
| | | acccctccaaccacagttttgcttctaaattgtaccccaacacacctagc |
| | | aaattgaaaccccaccagaagtcccccagatctggctttccggctattgc |
| | | tggcaagggggagtgactcccggcccattcaatccaggccccgcgtgttc |
| | | ctcaaacaagaagccacgtaaacataaaccgagcctccatgctgaccctt |
| | | gcccatcgaggtactcaatgttcacgtgatatccacacccagagggtcct |
| | | ggggtgggtgcatgagccccagaatgcaggcttgataaccgagaccctga |
| | | atcgggcagtgtccacaagggcggaggccagtcatgcatgttcgggccta |
| | | tggggccagcacccaacgccaaaactctccatcctcttcctcaatctcgc |
| | | tttctctctctctctctttttttttttttattttttttttttgcaaaagg |
| | | aggggagagggggtaaaaaaatgctgcactgtgcggctaggccggtgagt |
| | | gagcggcgcggagccaatcagcgctcgccgttccgaaagttgccttttat |
| | | ggctcgagtggccgctgtggcgtcctataaaacccggcggcgcaacgcgc |
| | | agccactgtcgagtccgcgtccacccgcgagcacaggcctttcgcagctc |
| | | tttcttcgccgctccacacccgccaccaggtaagcagggacaacaggccc |
| | | agccggccacagccctcccgtgggcagtgaccgcgctgcagggtcgcggg |
| | | ggacactcggcgcggacaccggggaaggctggagggtggtgccgggccgc |
| | | ggagcggacactttcagatccaactttcagtccagggtgtagacccttta |
| | | cagccgcattgccacggtgtagacaccggtggacccgctctggctcagag |
| | | cacgcggcttgggggaacccattagggtcgcagtgtgggcgctatgagag |
| | | ccgatgcagctttcgggtgttgaaccgtatctgcccaccttggggggagg |
| | | acacaaggtcgggagccaaacgccacgatcatgccttggtggcccatggg |
| | | tctttgtctaaaccggtttgcccatttggcttgccgggcgggcgggcgcg |
| | | gcgggcccggctcggccgggtgggggctgggttgccactgcgcttgcgcg |
| | | ctctatggctgggtattggggcgcgtgcacgctggggagggagcccttcc |
| | | tcttccccctctcccaagttaaacttgcgcgtgcgtattgagacttggag |
| | | cgcggccaccggggttgggcgagggcggggccgttgtccggaaggggcgg |
| | | ggtcgcagcggcttcggggcgcctgctcgcgcttcctgctgggtgtggtc |
| | | gcctcccgcgcgcgcactagccgcccgccggcggggcgaaggcggggctt |
| | | gcgcccgtttggggagggggcggaggcctggcttcctgccgtggggccgc |
| | | ctccggaccagcgtttgcctcttatggtaataacgcggccggcctgggct |
| | | tcctttgtcccctgagtttgggcgcgcgccccctggcggcccgaggccgc |
| | | ggcttgccggaagtgggcagggcggcagcggctgcgcctagtggcccgct |
| | | agtgaccgcgaccctcttttgtgccctgatatagttcgccgg |
| 5 | Hamster | ATGgccacctcagcaagttcccacttgaacaaaaacatcaagcaaatgta |
| | Glutamine | cttgtgcctgccccagggtgagaaagtccaagccatgtatatctgggttg |
| | synthetase | atggtactggagaaggactgcgctgcaaaacccgcaccctggactgtgag |
| | coding | cccaagtgtgtagaagagttacctgagtggaattttgatggctctagtac |
| | sequence | ctttcagtctgagggctccaacagtgacatgtatctcagccctgttgcca |
| | | tgtttcgggaccccttccgcagagatcccaacaagctggtgttctgtgaa |
| | | gttttcaagtacaaccggaagcctgcagagaccaatttaaggcactcgtg |
| | | taaacggataatggacatggtgagcaaccagcacccctggtttggaatgg |
| | | aacaggagtatactctgatgggaacagatgggcacccttttggttggcct |
| | | tccaatggctttcctgggccccaaggtccgtattactgtggtgtgggcgc |
| | | agacaaagcctatggcagggatatcgtggaggctcactaccgcgcctgct |
| | | tgtatgctggggtcaagattacaggaacaaatgctgaggtcatgcctgcc |
| | | cagtgggaatttcaaataggaccctgtgaaggaatccgcatgggagatca |
| | | tctctgggtggcccgtttcatcttgcatcgagtatgtgaagactttgggg |
| | | taatagcaacctttgaccccaagcccattcctgggaactggaatggtgca |
| | | ggctgccataccaactttagcaccaaggccatgcgggaggagaatggtct |
| | | gaagcacatcgaggaggccatcgagaaactaagcaagcggcaccggtacc |
| | | acattcgagcctacgatcccaaggggggcctggacaatgcccgtcgtctg |
| | | actgggttccacgaaacgtccaacatcaacgacttttctgctggtgtcgc |
| | | caatcgcagtgccagcatccgcattccccggactgtcggccaggagaaga |
| | | aaggttactttgaagaccgccgcccctctgccaattgtgacccctttgca |
| | | gtgacagaagccatcgtccgcacatgccttctcaatgagactggcgacga |
| | | gcccttccaatacaaaaacTAA |
| 6 | Hamster | MATSASSHLNKNIKQMYLCLPQGEKVQAMYIWVDGTGEGLRCKTRTLDCE |
| | Glutamine | PKCVEELPEWNFDGSSTFQSEGSNSDMYLSPVAMFRDPFRRDPNKLVFCE |
| | synthetase | VFKYNRKPAETNLRHSCKRIMDMVSNQHPWFGMEQEYTLMGTDGHPFGWP |
| | amino acid | SNGFPGPQGPYYCGVGADKAYGRDIVEAHYRACLYAGVKITGTNAEVMPA |
| | sequence | QWEFQIGPCEGIRMGDHLWVARFILHRVCEDFGVIATFDPKPIPGNWNGA |
| | | GCHTNFSTKAMREENGLKHIEEAIEKLSKRHRYHIRAYDPKGGLDNARRL |
| | | TGFHETSNINDFSAGVANRSASIRIPRTVGQEKKGYFEDRRPSANCDPFA |
| | | VTEAIVRTCLLNETGDEPFQYKN |
| 7 | CD52 | TTGgagcgcttcctcttcctcctactcaccatcagcctcctcgttttggt |
| | FLARE | acaaatacaaaccggactctccggacaaaacgacaccagccaaaccagca |
| | Reporter | gcccctcagcatccagcaacataagcggaggcattttccttttcttcgtc |
| | coding | gccaacgccataatccacctcttctgcttcagtTGA |
| | sequence | |
| 8 | CD52 | LERFLFLLLTISLLVLVQIQTGLSGQNDTSQTSSPSASSNISGGIFLFFV |
| | FLARE | ANAIIHLFCES |
| | Reporter | |
| | amino acid | |
| | sequence | |
| 9 | Ubiquitous | GGCCCTCCGCGCCTACAGCTCAAGCCACATCCGAAGGGGGAGGGAGCCGG |
| | chromatin | GAGCTGCGCGCGGGGCCGCCGGGGGGAGGGGTGGCACCGCCCACGCCGGG |
| | opening | CGGCCACGAAGGGCGGGGCAGCGGGCGCGCGCGCGGCGGGGGGAGGGGCC |
| | element | GGCGCCGCGCCCGCTGGGAATTGGGGCCCTAGGGGGAGGGCGGAGGCGCC |
| | (A2UCOE) | GACGACCGCGGCACTTACCGTTCGCGGCGTGGCGCCCGGTGGTCCCCAAG |
| | | GGGAGGGAAGGGGGAGGCGGGGCGAGGACAGTGACCGGAGTCTCCTCAGC |
| | | GGTGGCTTTTCTGCTTGGCAGCCTCAGCGGCTGGCGCCAAAACCGGACTC |
| | | CGCCCACTTCCTCGCCCGCCGGTGCGAGGGTGTGGAATCCTCCAGACGCT |
| | | GGGGGAGGGGGAGTTGGGAGCTTAAAAACTAGTACCCCTTTGGGACCACT |
| | | TTCAGCAGCGAACTCTCCTGTACACCAGGGGTCAGTTCCACAGACGCGGG |
| | | CCAGGGGTGGGTCATTGCGGCGTGAACAATAATTTGACTAGAAGTTGATT |
| | | CGGGTGTTTCCGGAAGGGGCCGAGTCAATCCGCCGAGTTGGGGCACGGAA |
| | | AACAAAAAGGGAAGGCTACTAAGATTTTTCTGGCGGGGGTTATCATTGGC |
| | | GTAACTGCAGGGACCACCTCCCGGGTTGAGGGGGCTGGATCTCCAGGCTG |
| | | CGGATTAAGCCCCTCCCGTCGGCGTTAATTTCAAACTGCGCGACGTTTCT |
| | | CACCTGCCTTCGCCAAGGCAGGGGCCGGGACCCTATTCCAAGAGGTAGTA |
| | | ACTAGCAGGACTCTAGCCTTCCGCAATTCATTGAGCGCATTTACGGAAGT |
| | | AACGTCGGGTACTGTCTCTGGCCGCAAGGGTGGGAGGAGTACGCATTTGG |
| | | CGTAAGGTGGGGCGTAGAGCCTTCCCGCCATTGGCGGCGGATAGGGCGTT |
| | | TACGCGACGGCCTGACGTAGCGGAAGACGCCTTAGTGGGGGGGAAGGTTC |
| | | TAGAAAAGCGGCGGCAGCGGCTCTAGCGGCAGTAGCAGCAGCGCCGGGTC |
| | | CCGTGCGGAGGTGCTCCTCGCAGAGTTGTTTCTCCAGCAGCGGCAGTTCT |
| | | CACTACAGCGCCAGGACGAGTCCGGTTCGTGTTCGTCCGCGGAGATCTCT |
| | | CTCATCTCGCTCGGCTGCGGGAAATCGGGCTGAAGCGACTGAGT |
| 10 | Chicken | gagggacagcccccccccaaagcccccagggatgtaattacgtccctccc |
| | hyper- | ccgctagggggcagcagcgagccgcccggggctccgctccggtccggcgc |
| | sensitivity | tccccccgcatccccgagccggcagcgtgcggggacagcccgggcacggg |
| | site 4 | gaaggtggcacgggatcgctttcctctgaacgcttctcgctgctctttga |
| | (CHS4) core | gcctgcagacacctggggggatacggggaaaa |
| | insulator | |
| 11 | Anti-Factor | ATGagagttccagctcagctgctgggactgctgctgttgtggttgcctgg |
| | Bb antibody | cgctagatgcgacatccagatgacccagtctccatccacactgtccgcct |
| | light chain | ctgtgggcgacagagtgaccatcacatgcaaggcctctcaggatgtgggc |
| | coding | accgccgttgcttggtatcagcagaagcctggcaaggcccctaagctgct |
| | sequence | gatctactgggcctccaccagacataccggcgtgcccgatagattctccg |
| | | gctctggctctggcaccgactttaccctgacaatcagctccctgcaggcc |
| | | gaggatttcgccgtgtacttctgccaccagcacagcagcaaccctctgac |
| | | ctttggccagggcaccaagctggaaatcaagcggacagtcgccgctcctt |
| | | ccgtgttcatcttcccaccttccgacgagcagctgaagtccggcacagct |
| | | tctgtcgtgtgcctgctgaacaacttctaccctcgggaagccaaggtgca |
| | | gtggaaggtggacaatgccctgcagtccggcaactcccaagagtctgtga |
| | | ccgagcaggactccaaggacagcacctacagcctgtcctccacactgacc |
| | | ctgtccaaggccgactacgagaagcacaaggtgtacgcctgcgaagtgac |
| | | ccatcagggcctgtctagccctgtgaccaagtctttcaaccggggcgagt |
| | | gcTGA |
| 12 | Anti-Factor | DIQMTQSPSTLSASVGDRVTITCKASQDVGTAVAWYQQKPGKAPKLLIYW |
| | Bb antibody | ASTRHTGVPDRFSGSGSGTDFTLTISSLQAEDFAVYFCHQHSSNPLTFGQ |
| | light chain | GTKLEIKRTVAAPSVFIFPPSDEQLKSGTASVVCLLNNFYPREAKVQWKV |
| | amino acid | DNALQSGNSQESVTEQDSKDSTYSLSSTLTLSKADYEKHKVYACEVTHQG |
| | sequence | LSSPVTKSFNRGEC |
| | (without | |
| | signal | |
| | sequence) | |
| 13 | Anti-Factor | ATGggatggtctttgatcctgctgttcctggtggccgtggctaccagagt |
| | Bb antibody | gcattctgaggtgcagctggtggaatctggcggcggacttgtgaaacctg |
| | heavy chain | gcggctctctgagactgtcttgtgccgcttctggcttcaccttctccaac |
| | coding | tacgccatgtcctgggtccgacaggctcctggcaaaagactggaatgggt |
| | sequence | cgccaccatctccaaccggggctcctacacctactatcccgactccgtga |
| | | agggcagattcaccatcagccgggacaacgccaagaactccctgtacctg |
| | | cagatgaacagcctgagagccgaggacaccgctctgtactactgcgccag |
| | | agaacggcccatggattattggggccagggcacactggtcaccgtgtcct |
| | | ctgcttctaccaagggacccagcgtgttccctctggctccttgctccaga |
| | | tccacctccgagtctacagctgctctgggctgcctggtcaaggactactt |
| | | tcctgagcctgtgaccgtgtcttggaactctggcgctctgacatccggcg |
| | | tgcacacctttccagctgtgctgcaatcctccggcctgtactctctgtcc |
| | | tccgtcgtgaccgtgccttctagctctctgggcaccaagacctacacctg |
| | | taatgtggaccacaagccttccaacaccaaggtggacaagcgcgtggaat |
| | | ctaagtacggccctccttgtcctccatgtccagctccagagtttgaaggc |
| | | ggcccttccgtgttcctgtttcctccaaagcctaaggacaccctgatgat |
| | | ctctcggacccctgaagtgacctgcgtggtggtggatgtgtcccaagagg |
| | | atcccgaggtgcagttcaattggtacgtggacggcgtggaagtgcacaat |
| | | gccaagaccaagcctagagaggaacagttcaactccacctatagagtggt |
| | | gtccgtgctgaccgtgctgcaccaggattggctgaacggcaaagagtaca |
| | | agtgcaaggtgtccaacaagggcctgcctagctccatcgagaaaaccatc |
| | | agcaaggccaagggccagcctcgggaacctcaggtttacaccctgcctcc |
| | | aagccaagaggaaatgaccaagaaccaggtgtccctgacctgcctcgtga |
| | | agggattctacccctccgatatcgccgtggaatgggagtctaatggacag |
| | | cccgagaacaactacaagacaacccctcctgtgctggactccgacggcag |
| | | cttcttcctgtattctcgcctgaccgtggacaagtccagatggcaagagg |
| | | gcaacgtgttctcctgcagcgtgctgcatgaggccctgcactctcactac |
| | | acccagaagtccctgtctctgtccctgggcaaaTGA |
| 14 | Anti-Factor | EVQLVESGGGLVKPGGSLRLSCAASGFTFSNYAMSWVRQAPGKRLEWVAT |
| | Bb antibody | ISNRGSYTYYPDSVKGRFTISRDNAKNSLYLQMNSLRAEDTALYYCARER |
| | heavy chain | PMDYWGQGTLVTVSSASTKGPSVFPLAPCSRSTSESTAALGCLVKDYFPE |
| | amino acid | PVTVSWNSGALTSGVHTFPAVLQSSGLYSLSSVVTVPSSSLGTKTYTCNV |
| | sequence | DHKPSNTKVDKRVESKYGPPCPPCPAPEFEGGPSVFLFPPKPKDTLMISR |
| | (without | TPEVTCVVVDVSQEDPEVQFNWYVDGVEVHNAKTKPREEQFNSTYRVVSV |
| | signal | LTVLHQDWLNGKEYKCKVSNKGLPSSIEKTISKAKGQPREPQVYTLPPSQ |
| | sequence) | EEMTKNQVSLTCLVKGFYPSDIAVEWESNGQPENNYKTTPPVLDSDGSFF |
| | | LYSRLTVDKSRWQEGNVFSCSVLHEALHSHYTQKSLSLSLGK |
| 15 | Anti- | atgggctggtcctgcatcatcctgtttctggtggctaccgctaccggcgt |
| | CD40L | gcactccgatatcgtgatgacccagtctcctagcttcctgtccgcctctg |
| | antibody | tgggcgacagagtgacaattacctgcaaggcctcctccaacctgggccat |
| | light chain | gctgtggcttggtatcagcagaagcctggcaagtcccctaagctgctgat |
| | coding | ctactccgcctccaacagatacacaggcgtgcccgacagattctccggct |
| | sequence | ctggctctggcacagactttaccctgacaatctccagcctgcagcctgag |
| | | gacttcgccgactacttctgccagcagtacgacgactacccctacacctt |
| | | tggcggaggcaccaagctggaaatcaagcggacagtggccgctccttccg |
| | | tgttcatcttcccaccttccgacgagcagctgaagtctggcaccgcttct |
| | | gtcgtgtgcctgctgaacaacttctaccctcgggaagccaaggtgcagtg |
| | | gaaggtggacaatgccctgcagtccggcaactcccaagagtctgtgaccg |
| | | agcaggactccaaggacagcacctacagcctgtcctccacactgaccctg |
| | | tccaaggccgactacgagaagcacaaggtgtacgcctgcgaagtgaccca |
| | | tcagggcctgtctagccctgtgaccaagtctttcaaccggggcgagtgct |
| | | ga |
| 16 | Anti- | DIVMTQSPSFLSASVGDRVTITCKASSNLGHAVAWYQQKPGKSPKLLIYS |
| | CD40L | ASNRYTGVPDRFSGSGSGTDFTLTISSLQPEDFADYFCQQYDDYPYTFGG |
| | antibody | GTKLEIKRTVAAPSVFIFPPSDEQLKSGTASVVCLLNNFYPREAKVQWKV |
| | light chain | DNALQSGNSQESVTEQDSKDSTYSLSSTLTLSKADYEKHKVYACEVTHQG |
| | amino acid | LSSPVTKSFNRGEC* |
| | sequence | |
| | (without | |
| | signal | |
| | sequence) | |
| 17 | Anti- | atgggctggtcctgcatcatcctgtttctggtggctaccgctaccggcgt |
| | CD40L | gcactctgaagttcagctgcaagagtctggccctggcctggtcaagcctt |
| | antibody | ccgaaacactgtctctgacctgcaccgtgtccggcgactctatcaccaac |
| | heavy chain | ggcttttggatctggatccggaagcctcctggcaacaagctcgagtatat |
| | coding | gggctacatctcctactccggctccacctactacaaccccagcctgaagt |
| | sequence | cccggatctccatctccagagacacctccaagaaccagttctccctgaag |
| | | ctgtcctccgtgaccgctgctgatacaggcgtgtactactgcgcctaccg |
| | | gtcctatggcagaaccccttactacttcgactactggggccagggcacca |
| | | cactgacagtgtcctctgcttccaccaagggacccagcgttttccctctg |
| | | gctccatcctccaagtctacctctggcggaacagctgctctgggctgtct |
| | | ggtcaaggactacttccctgagcctgtgaccgtgtcctggaattctggcg |
| | | ctctgacatctggcgtgcacacctttccagctgtgctgcagtcctccggc |
| | | ctgtactctctgtcctctgtcgtgaccgtgccttccagctctctgggaac |
| | | ccagacctacatctgcaatgtgaaccacaagcctagcaacaccaaggtgg |
| | | acaagaaggccgagcctaagtcctgcgacaagacccacacctgtcctcca |
| | | tgtcctgctccagaactgctcggcggaccttccgtgttcctgtttcctcc |
| | | aaagcctaaggacaccctgatgatctctcggacccctgaagtgacctgcg |
| | | tggtggtggatgtgtctcaccgggatccagaagtgaagttcaattggtac |
| | | gtggacggcgtggaagtgcacaacgccaagaccaagcctagagaggaaca |
| | | gtacaacagcacctacagagtggtgtccgtgctgaccgtgctgcaccagg |
| | | attggctgaacggcaaagagtacaagtgcgccgtgtccaacaaggccctg |
| | | cctgctcctatcgaaaagaccatctccaaggctaagggccagcctcggga |
| | | acctcaggtgtacacattgcctccatctcgggacgagctgaccaagaatc |
| | | aggtgtccctgacctgtctcgtgaagggcttctacccctccgatatcgcc |
| | | gtggaatgggagtccaatggccagcctgagaacaactacaagacaacccc |
| | | tcctgtgctggactccgacggctcattcttcctgtactccaagctgacag |
| | | tggacaagtccagatggcagcagggcaacgtgttctcctgctccgtgatg |
| | | cacgaggccctgcacaatcactacacccagaagtccctgtctctgtcccc |
| | | tggcaaatga |
| 18 | Anti- | EVQLQESGPGLVKPSETLSLTCTVSGDSITNGFWIWIRKPPGNKLEYMGY |
| | CD40L | ISYSGSTYYNPSLKSRISISRDTSKNQFSLKLSSVTAADTGVYYCAYRSY |
| | antibody | GRTPYYFDYWGQGTTLTVSSASTKGPSVFPLAPSSKSTSGGTAALGCLVK |
| | heavy chain | DYFPEPVTVSWNSGALTSGVHTFPAVLQSSGLYSLSSVVTVPSSSLGTQT |
| | amino acid | YICNVNHKPSNTKVDKKAEPKSCDKTHTCPPCPAPELLGGPSVFLFPPKP |
| | sequence | KDTLMISRTPEVTCVVVDVSHRDPEVKFNWYVDGVEVHNAKTKPREEQYN |
| | (without | STYRVVSVLTVLHQDWLNGKEYKCAVSNKALPAPIEKTISKAKGQPREPQ |
| | signal | VYTLPPSRDELTKNQVSLTCLVKGFYPSDIAVEWESNGQPENNYKTTPPV |
| | sequence) | LDSDGSFFLYSKLTVDKSRWQQGNVFSCSVMHEALHNHYTQKSLSLSPGK |
| 19 | Anti-α- | ATGGGCTGGTCCTGCATCATTCTGTTTCTGGTGGCTACCGCTACCGGCGT |
| | synuclein/ | GCACTCCGATATCGTGATGACCCAGACACCTCTGAGCCTGTCTGTGACAC |
| | IGF1R | CTGGCCAGCCTGCCTCCATCTCCTGCAGATCTTCTCAGTCCCTGGTGCAC |
| | antibody | TCCAACGGCAACACCTACCTGCACTGGTATCTGCAGAAGCCCGGACAGTC |
| | light chain | TCCCCAGCTGCTGATCTACAAGGTGTCCAACAGATTCTCTGGCGTGCCCG |
| | coding | ACAGATTCAGCGGCTCTGGCTCTGGCACCGACTTCACCCTGAAGATCTCT |
| | sequence | AGAGTGGAAGCCGAGGACGTGGGCGTGTACTACTGCTCTCAGTCTACCCA |
| | | CGTGCCAAGAACCTTTGGCCAGGGCACCAAGCTGGAAATCAAGAGAACCG |
| | | TGGCCGCTCCTTCCGTGTTCATCTTCCCACCATCTGACGAGCAGCTGAAG |
| | | TCCGGCACAGCTTCTGTCGTGTGCCTGCTGAACAACTTCTACCCTCGGGA |
| | | AGCCAAGGTGCAGTGGAAGGTGGACAATGCCCTGCAGTCCGGCAACTCCC |
| | | AAGAGTCTGTGACCGAGCAGGACTCCAAGGACTCTACCTACTCTCTGTCC |
| | | TCCACACTGACCCTGTCCAAGGCCGACTACGAGAAGCACAAGGTGTACGC |
| | | CTGTGAAGTGACCCACCAGGGCCTGTCTAGCCCTGTGACCAAGTCTTTCA |
| | | ACCGGGGCGAGTGCTGA |
| 20 | Anti-α- | DIVMTQTPLSLSVTPGQPASISCRSSQSLVHSNGNTYLHWYLQKPGQSPQ |
| | synuclein/ | LLIYKVSNRFSGVPDRFSGSGSGTDFTLKISRVEAEDVGVYYCSQSTHVP |
| | IGF1R | RTFGQGTKLEIKRTVAAPSVFIFPPSDEQLKSGTASVVCLLNNFYPREAK |
| | antibody | VQWKVDNALQSGNSQESVTEQDSKDSTYSLSSTLTLSKADYEKHKVYACE |
| | light chain | VTHQGLSSPVTKSFNRGEC |
| | amino acid | |
| | sequence | |
| | (without | |
| | signal | |
| | sequence) | |
| 21 | Anti-α- | ATGGGCTGGTCCTGCATCATTCTGTTTCTGGTGGCTACCGCTACCGGCGT |
| | synuclein/ | GCACTCTCAGGTTCAGTTGGTTCAGTCTGGCGCCGAAGTGAAGAAACCTG |
| | IGF1R | GCGCTTCTGTGAAGGTGTCCTGCAAGGCCTCTGGCTACACCTTTACCAAC |
| | antibody | TACCTGATCGAGTGGGTCCGACAGGCTCCTGGACAAGGACTGGAATGGAT |
| | heavy chain | GGGCGTGATCAATCCTGGCTCTGGCGGCACCAACTACAACGAGAAGTTCA |
| | 1 | AGGGCAGAGTGACCATGACCAGAGACACCTCCATCTCCACCGCCTACATG |
| | coding | GAACTGTCCCGGCTGAGATCTGACGACACCGCCGTGTACTACTGCGCCTC |
| | sequence | TGGCAACTACGATACCTATTGGGGCCAGGGCACACTGGTCACCGTGTCCT |
| | | CTGCTTCTACCAAGGGACCCTCTGTGTTCCCTCTGGCTCCTTCCAGCAAG |
| | | TCCACATCTGGTGGAACCGCTGCTCTGGGCTGCCTCGTGAAGGATTACTT |
| | | TCCTGAGCCTGTGACCGTGTCTTGGAACTCTGGTGCTCTGACCTCTGGCG |
| | | TGCACACCTTTCCAGCTGTGCTGCAGTCCTCTGGCCTGTACTCTCTGTCC |
| | | TCTGTCGTGACCGTGCCTTCTAGCTCTCTGGGCACCCAGACCTACATCTG |
| | | CAACGTGAACCACAAGCCTTCCAACACCAAGGTGGACAAGAAGGTGGAAC |
| | | CCAAGTCCTGCGACAAGACCCACACCTGTCCTCCATGTCCTGCTCCAGAA |
| | | CTGCTCGGCGGTCCCTCCGTTTTCCTGTTTCCACCTAAGCCTAAGGACAC |
| | | CCTGATGATCTCTCGGACCCCTGAAGTGACCTGCGTGGTGGTGGATGTGT |
| | | CTCACGAGGACCCAGAAGTGAAGTTCAATTGGTACGTGGACGGCGTGGAA |
| | | GTGCACAACGCCAAGACCAAGCCTAGAGAGGAACAGTACAACTCCACCTA |
| | | CAGAGTGGTGTCCGTGCTGACCGTGCTGCACCAGGATTGGCTGAACGGCA |
| | | AAGAGTACAAGTGCAAGGTGTCCAACAAGGCACTGCCCGCTCCTATCGAA |
| | | AAGACCATCTCCAAGGCTAAGGGCCAGCCTCGGGAACCTCAGGTTTACAC |
| | | ACTGCCTCCATCTAGGGACGAGCTGACCAAGAATCAGGTGTCCCTGTGGT |
| | | GCCTGGTCAAGGGCTTCTACCCTTCCGATATCGCCGTGGAATGGGAGTCC |
| | | AATGGCCAGCCTGAGAACAACTACAAGACAACCCCTCCTGTGCTGGACTC |
| | | CGACGGCTCATTCTTCCTGTACTCCAAGCTGACAGTGGACAAGTCTCGGT |
| | | GGCAGCAGGGCAACGTGTTCTCTTGTAGTGTGCTGCACGAGGCCCTGCAC |
| | | AACCACTACACACAGAAGTCCCTGTCTCTGTCCCCTGGCAAATGA |
| 22 | Anti-α- | QVQLVQSGAEVKKPGASVKVSCKASGYTFTNYLIEWVRQAPGQGLEWMGV |
| | synuclein/I | INPGSGGTNYNEKFKGRVTMTRDTSISTAYMELSRLRSDDTAVYYCASGN |
| | GF1R | YDTYWGQGTLVTVSSASTKGPSVFPLAPSSKSTSGGTAALGCLVKDYFPE |
| | antibody | PVTVSWNSGALTSGVHTFPAVLQSSGLYSLSSVVTVPSSSLGTQTYICNV |
| | heavy chain | NHKPSNTKVDKKVEPKSCDKTHTCPPCPAPELLGGPSVFLFPPKPKDTLM |
| | 1 | ISRTPEVTCVVVDVSHEDPEVKFNWYVDGVEVHNAKTKPREEQYNSTYRV |
| | amino acid | VSVLTVLHQDWLNGKEYKCKVSNKALPAPIEKTISKAKGQPREPQVYTLP |
| | sequence | PSRDELTKNQVSLWCLVKGFYPSDIAVEWESNGQPENNYKTTPPVLDSDG |
| | (without | SFFLYSKLTVDKSRWQQGNVFSCSVLHEALHNHYTQKSLSLSPGK* |
| | signal | |
| | sequence) | |
| 23 | Anti-α- | ATGGGCTGGTCCTGCATCATTCTGTTTCTGGTGGCTACCGCTACCGGCGT |
| | synuclein/ | GCACTCTCAGGTTCAGTTGGTTCAGTCTGGCGCCGAAGTGAAGAAACCTG |
| | IGF1R | GCGCTTCTGTGAAGGTGTCCTGCAAGGCCTCTGGCTACACCTTTACCAAC |
| | antibody | TACCTGATCGAGTGGGTCCGACAGGCTCCTGGACAAGGACTGGAATGGAT |
| | heavy chain | GGGCGTGATCAATCCTGGCTCTGGCGGCACCAACTACAACGAGAAGTTCA |
| | 2 | AGGGCAGAGTGACCATGACCAGAGACACCTCCATCTCCACCGCCTACATG |
| | coding | GAACTGTCCCGGCTGAGATCTGACGACACCGCCGTGTACTACTGCGCCTC |
| | sequence | TGGCAACTACGATACCTATTGGGGCCAGGGCACACTGGTCACCGTGTCCT |
| | | CTGCTTCTACCAAGGGACCCTCTGTGTTCCCTCTGGCTCCTTCCAGCAAG |
| | | TCCACATCTGGTGGAACCGCTGCTCTGGGCTGCCTCGTGAAGGATTACTT |
| | | TCCTGAGCCTGTGACCGTGTCTTGGAACTCTGGTGCTCTGACCTCTGGCG |
| | | TGCACACCTTTCCAGCTGTGCTGCAGTCCTCTGGCCTGTACTCTCTGTCC |
| | | TCTGTCGTGACCGTGCCTTCTAGCTCTCTGGGCACCCAGACCTACATCTG |
| | | CAACGTGAACCACAAGCCTTCCAACACCAAGGTGGACAAGAAGGTGGAAC |
| | | CCAAGTCCTGCGACAAGACCCACACCTGTCCTCCATGTCCTGCTCCAGAA |
| | | CTGCTCGGCGGTCCCTCCGTTTTCCTGTTTCCACCTAAGCCTAAGGACAC |
| | | CCTGATGATCTCTCGGACCCCTGAAGTGACCTGCGTGGTGGTGGATGTGT |
| | | CTCACGAGGACCCAGAAGTGAAGTTCAATTGGTACGTGGACGGCGTGGAA |
| | | GTGCACAACGCCAAGACCAAGCCTAGAGAGGAACAGTACAACTCCACCTA |
| | | CAGAGTGGTGTCCGTGCTGACCGTGCTGCACCAGGATTGGCTGAACGGCA |
| | | AAGAGTACAAGTGCAAGGTGTCCAACAAGGCACTGCCCGCTCCTATCGAA |
| | | AAGACCATCTCCAAGGCTAAGGGCCAGCCTCGGGAACCTCAGGTTTACAC |
| | | ACTGCCTCCATCTAGGGACGAGCTGACCAAGAACCAGGTGTCCCTGTCTT |
| | | GTGCCGTGAAGGGCTTCTACCCTTCCGATATCGCCGTGGAATGGGAGTCC |
| | | AATGGCCAGCCTGAGAACAACTACAAGACAACCCCTCCTGTGCTGGACTC |
| | | CGACGGCTCATTCTTCCTGGTGTCCAAGCTGACAGTGGACAAGTCCAGAT |
| | | GGCAGCAGGGCAACGTGTTCTCCTGTAGCGTGCTGCATGAGGCCCTGCAC |
| | | AACCACTACACACAGAAGTCTCTGTCTCTGAGCCCTGGCAAAGGTGGCGG |
| | | AGGATCTGGCGGAGGTGGAAGCGGCGGAGGCGGATCTCAATCTGTTCTGA |
| | | CTCAGCCTCCTTCCGCTTCTGGCACACCTGGCCAGAGAGTGACAATCTCT |
| | | TGCACCGGCTCCAGCTCCAACATCGGCTCCAACGATGTGTCCTGGTATCA |
| | | GCAGCTGCCTGGCACAGCCCCTAAACTGCTGATCTACGCCAATGTGAACA |
| | | GACCCTCCGGCGTGCCCGATAGATTCTCTGGCTCTAAGTCTGGCACCAGC |
| | | GCCAGCCTGGCTATCTCTGGACTGAGATCCGAGGACGAGGCCGACTACTA |
| | | TTGTGGCACTTGGGCCGGATCTCTGAACGCCTATGTGTTCGGCTGTGGCA |
| | | CAAAGCTGACTGTTCTTGGAGGCGGTGGTAGTGGTGGTGGTGGTTCTGGT |
| | | GGTGGCGGAAGCGGAGGTGGTGGATCTGAAGTTCAGCTGTTGGAATCTGG |
| | | CGGCGGACTTGTTCAGCCTGGTGGCTCTTTGAGACTGTCTTGCGCTGCCT |
| | | CCGGCTTCACCTTCTCCTCTTACGACATGTCCTGGGTTCGACAAGCCCCA |
| | | GGCAAGTGTCTGGAATGGGTTTCCGCCATCTCCTACGATAACGCCAACAC |
| | | CTACTACGCCGACAGCGTGAAGGGAAGATTCACCATCTCTCGGGACAACA |
| | | GCAAGAACACCCTGTACCTGCAGATGAACTCCCTGAGAGCTGAGGATACA |
| | | GCTGTCTACTACTGTGCCAAGGGCGTGCTGACAACACTGATGAATTGGTT |
| | | CGACTACTGGGGACAGGGAACCCTCGTGACAGTGTCCTCTTGA |
| 24 | Anti-α- | QVQLVQSGAEVKKPGASVKVSCKASGYTFTNYLIEWVRQAPGQGLEWMGV |
| | synuclein/ | INPGSGGTNYNEKFKGRVTMTRDTSISTAYMELSRLRSDDTAVYYCASGN |
| | IGF1R | YDTYWGQGTLVTVSSASTKGPSVFPLAPSSKSTSGGTAALGCLVKDYFPE |
| | antibody | PVTVSWNSGALTSGVHTFPAVLQSSGLYSLSSVVTVPSSSLGTQTYICNV |
| | heavy chain | NHKPSNTKVDKKVEPKSCDKTHTCPPCPAPELLGGPSVFLFPPKPKDTLM |
| | 2 | ISRTPEVTCVVVDVSHEDPEVKFNWYVDGVEVHNAKTKPREEQYNSTYRV |
| | amino acid | VSVLTVLHQDWLNGKEYKCKVSNKALPAPIEKTISKAKGQPREPQVYTLP |
| | sequence | PSRDELTKNQVSLSCAVKGFYPSDIAVEWESNGQPENNYKTTPPVLDSDG |
| | (without | SFFLVSKLTVDKSRWQQGNVFSCSVLHEALHNHYTQKSLSLSPGKGGGGS |
| | signal | GGGGSGGGGSQSVLTQPPSASGTPGQRVTISCTGSSSNIGSNDVSWYQQL |
| | sequence) | PGTAPKLLIYANVNRPSGVPDRFSGSKSGTSASLAISGLRSEDEADYYCG |
| | | TWAGSLNAYVFGCGTKLTVLGGGGSGGGGSGGGGSGGGGSEVQLLESGGG |
| | | LVQPGGSLRLSCAASGFTFSSYDMSWVRQAPGKCLEWVSAISYDNANTYY |
| | | ADSVKGRFTISRDNSKNTLYLQMNSLRAEDTAVYYCAKGVLTTLMNWEDY |
| | | WGQGTLVTVSS* |
| 25 | 5′ ITR | ccctagaaagataatcatattgtgacgtacgttaaagataatcatgcgta |
| | | aaattgacgcatgtgttttatcggtctgtatatcgaggtttatttattaa |
| | | tttgaatagatattaagttttattatatttacacttacatactaataata |
| | | aattcaacaaacaatttatttatgtttatttatttattaaaaaaaaacaa |
| | | aaactcaaaatttcttctataaagtaacaaaactttta |
| 26 | 3′ ITR and | gatatctataacaagaaaatatatatataataagttatcacgtaagtaga |
| | internal | acatgaaataacaatataattatcgtatgagttaaatcttaaaagtcacg |
| | domain | taaaagataatcatgcgtcattttgactcacgcggtcgttatagttcaaa |
| | | atcagtgacacttaccgcattgacaagcacgcctcacgggagctccaagc |
| | | ggcgactgagatgtcctaaatgcacagcgacggattcgcgctatttagaa |
| | | agagagagcaatatttcaagaatgcatgcgtcaattttacgcagactatc |
| | | tttctaggg |
| 27 | Heavy chain | 5′-CTCCAAAGCCTAAGGACACC-3′ |
| | forward | |
| | primer | |
| 28 | Heavy chain | 5′-CGTACCAATTGAACTGCACC-3′ |
| | reverse | |
| | primer | |
| 29 | Heavy chain | 5′-CGGACCCCTGAAGTGACCTGCGTGG-3′ |
| | probe | |
| 30 | Light chain | 5′-CCCAAGAGTCTGTGACCGA-3′ |
| | forward | |
| | primer | |
| 31 | Light chain | 5′-GTACACCTTGTGCTTCTCGT-3′ |
| | reverse | |
| | primer | |
| 32 | Light chain | 5′-CCACACTGACCCTGTCCAAGGCCGA-3′ |
| | probe | |
| 33 | GAPDH | 5′-TCAAGAAGGTGGTGAAGCAG-3′ |
| | Forward | |
| | Primer | |
| 34 | GAPDH | 5′-CAAAGGTGGAAGAGTGGGAG-3′ |
| | Reverse | |
| | Primer | |
| 35 | GAPDH | 5′-TGGGCTACACCGAGGACCAGGTTGT-3′ |
| | Probe | |
| 36 | Cabbage | MGSSLDDEHILSALLQSDDELVGEDSDSEISDHVSEDDVQSDTEEAFIDE |
| | looper moth | VHEVQPTSSGSEILDEQNVIEQPGSSLASNKILTLPQRTIRGKNKHCWST |
| | transposase | SKSTRRSRVSALNIVRSQRGPTRMCRNIYDPLLCFKLFFTDEIISEIVKW |
| | sequence 1 | TNAEISLKRRESMTGATFRDTNEDEIYAFFGILVMTAVRKDNHMSTDDLF |
| | | DRSLSMVYVSVMSRDREDFLIRCLRMDDKSIRPTLRENDVETPVRKIWDL |
| | | FIHQCIQNYTPGAHLTIDEQLLGFRGRCPFRMYIPNKPSKYGIKILMMCD |
| | | SGTKYMINGMPYLGRGTQTNGVPLGEYYVKELSKPVRGSCRNITCDNWFT |
| | | SIPLAKNLLQEPYKLTIVGTVRSNKREIPEVLKNSRSRPVGTSMFCFDGP |
| | | LTLVSYKPKPAKMVYLLSSCDEDASINESTGKPQMVMYYNQTKGGVDTLD |
| | | QMCSVMTCSRKTNRWPMALLYGMINIACINSFIIYSHNVSSKGEKVQSRE |
| | | KFMRNLYMSLTSSFMRKRLEAPTLKRYLRDNISNILPNEVPGTSDDSTEE |
| | | PVTKKRTYCT YCPSKIRRKANASCKKCKKVICREHNIDMCQSCF |
| 37 | Cabbage | MGSSLDDEHILSALLQSDDELVGEDSDSEVSDHVSEDDVQSDTEEAFIDE |
| | looper moth | VHEVQPTSSGSEILDEQNVIEQPGSSLASNRILTLPQRTIRGKNKHCWST |
| | transposase | SKSTRRSRVSALNIVRSQRGPTRMCRNIYDPLLCFKLEFTDEIISEIVKW |
| | sequence 2 | TNAEISLKRRESMTSATERDTNEDEIYAFFGILVMTAVRKDNHMSTDDLF |
| | | DRSLSMVYVSVMSRDREDFLIRCLRMDDKSIRPTLRENDVETPVRKIWDL |
| | | FIHQCIQNYTPGAHLTIDEQLLGFRGRCPFRVYIPNKPSKYGIKILMMCD |
| | | SGTKYMINGMPYLGRGTQTNGVPLGEYYVKELSKPVHGSCRNITCDNWFT |
| | | SIPLAKNLLQEPYKLTIVGTVRSNKREIPEVLKNSRSRPVGTSMFCFDGP |
| | | LTLVSYKPKPAKMVYLLSSCDEDASINESTGKPQMVMYYNQTKGGVDTLD |
| | | QMCSVMTCSRKTNRWPMALLYGMINIACINSFIIYSHNVSSKGEKVQSRK |
| | | KFMRNLYMSLTSSFMRKRLEAPTLKRYLRDNISNILPKEVPGTSDDSTEE |
| | | PVMKKRTYCTYCPSKIRRKANASCKKCKKVICREHNIDMCQSCF |
| 38 | Human | MTTSASSHLNKGIKQVYMSLPQGEKVQAMYIWIDGTGEGLRCKTRTLDSE |
| | glutamine | PKCVEELPEWNEDGSSTLQSEGSNSDMYLVPAAMERDPFRKDPNKLVLCE |
| | synthase | VFKYNRRPAETNLRHTCKRIMDMVSNQHPWFGMEQEYTLMGTDGHPFGWP |
| | | SNGFPGPQGPYYCGVGADRAYGRDIVEAHYRACLYAGVKIAGTNAEVMPA |
| | | QWEFQIGPCEGISMGDHLWVARFILHRVCEDFGVIATFDPKPIPGNWNGA |
| | | GCHTNFSTKAMREENGLKYIEEAIEKLSKRHQYHIRAYDPKGGLDNARRL |
| | | TGFHETSNINDESAGVANRSASIRIPRTVGQEKKGYFEDRRPSANCDPFS |
| | | VTEALIRTCLLNETGDEPFQYKN |
| 39 | Anti-IL-1R3 | atgggctggtcctgcatcatcctgtttctggtggctaccgctaccggcgt |
| | antibody | gcactctgatgtgcagatgacccagtctccttccagcctgtctgcctctg |
| | light chain | tgggcgacagagtgaccatcacctgtcaggccagccagtctatctactcc |
| | coding | ttcctgtcctggtatcagcagaagcccggccaggctcctaagctgctgat |
| | sequence | ctacgctgcctccgacctggaatctggcgtgccctctagattctccggct |
| | | ctggctctggcaccgactttaccctgacaatctccagcctgcagcctgag |
| | | gacttcgccacctactactgccagtccaactacatcatcgactacggcgc |
| | | ctttggccagggcaccaaggtggtcatcaagagaacagtggccgctcctt |
| | | ccgtgttcatcttcccaccttccgacgagcagctgaagtccggcacagct |
| | | tctgtcgtgtgcctgctgaacaacttctaccctcgggaagccaaggtgca |
| | | gtggaaggtggacaatgccctgcagtccggcaactcccaagagtctgtga |
| | | ccgagcaggactccaaggacagcacctatagcctgtcctccacactgacc |
| | | ctgtccaaggccgactacgagaagcacaaggtgtacgcctgcgaagtgac |
| | | ccatcagggcctgtctagtcccgtgaccaagtctttcaaccggggcgagt |
| | | gttga |
| 40 | Anti-IL-1R3 | DVQMTQSPSSLSASVGDRVTITCQASQSIYSFLSWYQQKPGQAPKLLIYA |
| | antibody | ASDLESGVPSRFSGSGSGTDFTLTISSLQPEDFATYYCQSNYIIDYGAFG |
| | light chain | QGTKVVIKRTVAAPSVFIFPPSDEQLKSGTASVVCLLNNFYPREAKVQWK |
| | amino acid | VDNALQSGNSQESVTEQDSKDSTYSLSSTLTLSKADYEKHKVYACEVTHQ |
| | sequence | GLSSPVTKSFNRGEC |
| | (without | |
| | signal | |
| | sequence) | |
| 41 | Anti-IL-1R3 | atgggctggtcctgcatcatcctgtttctggtggctaccgctaccggcgt |
| | antibody | gcactctgaagtgcagctggaagaatctggcggcagactggtgcagcctg |
| | heavy chain | gcacatctctgagactgtcttgtgccgtgtccggcttctccctgtcctcc |
| | coding | tacgatatgtcctgggtccgacaggctcctggcaaaggactggaatgggt |
| | sequence | gtccaccatctacatcggcggaacaaccgcctacgcctcctggcctaagg |
| | | gcagattcaccatctccaagaccaactccaagaacaccctgtacctgcag |
| | | atgaactccctgagagccgaggataccgccgtgtacttctgcgctagact |
| | | gcagggcgccaactactacaactctctggctctgtggggccagggcacac |
| | | tggttacagtgtcctccgcttccaccaagggaccctctgtgtttcctctg |
| | | gctccttgctccagatccacctccgagtctacagctgctctgggctgcct |
| | | ggtcaaggactactttcctgagcctgtgaccgtgtcctggaactctggcg |
| | | ctctgacatctggcgtgcacacctttccagctgtgctgcagtcctccggc |
| | | ctgtactctctgtcctctgtcgtgaccgtgccttcctctagcctgggcac |
| | | caagacctacacctgtaatgtggaccacaagccttccaacaccaaggtgg |
| | | acaagcgcgtggaatctaagtacggccctccttgtcctccatgtcctgct |
| | | ccagaagctgctggcggcccatcagtgtttctgttccctccaaagcctaa |
| | | ggacaccctgatgatctctcggacccctgaagtgacctgcgtggtggtgg |
| | | atgtgtcccaagaggatcccgaggtgcagttcaattggtacgtggacggc |
| | | gtggaagtgcacaacgctaagaccaagcctagagaggaacagttcaactc |
| | | cacctacagagtggtgtccgtgctgaccgtgctgcatcaggattggctga |
| | | acggcaaagagtacaagtgcaaggtgtccaacaagggcctgccttccagc |
| | | atcgaaaagaccatcagcaaggccaagggccagcctagggaaccccaggt |
| | | ttacaccctgcctccaagccaagaggaaatgaccaagaaccaggtgtccc |
| | | tgacctgcctcgtgaagggcttctacccttccgatatcgccgtggaatgg |
| | | gagagcaatggccagcctgagaacaactacaagacaacccctcctgtgct |
| | | ggactccgacggcagcttcttcctgtattcccgcctgaccgtggacaagt |
| | | ccagatggcaagagggcaacgtgttctcctgctccgtgatgcacgaggcc |
| | | ctgcacaatcactacacccagaagtccctgtctctgtccctgggctaa |
| 42 | Anti-IL-1R3 | EVQLEESGGRLVQPGTSLRLSCAVSGFSLSSYDMSWVRQAPGKGLEWVST |
| | antibody | IYIGGTTAYASWPKGRFTISKTNSKNTLYLQMNSLRAEDTAVYFCARLQG |
| | heavy chain | ANYYNSLALWGQGTLVTVSSASTKGPSVFPLAPCSRSTSESTAALGCLVK |
| | amino acid | DYFPEPVTVSWNSGALTSGVHTFPAVLQSSGLYSLSSVVTVPSSSLGTKT |
| | sequence | YTCNVDHKPSNTKVDKRVESKYGPPCPPCPAPEAAGGPSVFLFPPKPKDT |
| | (without | LMISRTPEVTCVVVDVSQEDPEVQFNWYVDGVEVHNAKTKPREEQFNSTY |
| | signal | RVVSVLTVLHQDWLNGKEYKCKVSNKGLPSSIEKTISKAKGQPREPQVYT |
| | sequence) | LPPSQEEMTKNQVSLTCLVKGFYPSDIAVEWESNGQPENNYKTTPPVLDS |
| | | DGSFFLYSRLTVDKSRWQEGNVFSCSVMHEALHNHYTQKSLSLSLG |

1. An expression construct for expressing a recombinant antibody in a mammalian host cell, comprising a polycistronic expression cassette flanked by a pair of inverted terminal repeats (ITR) recognizable by a transposase, wherein the expression cassette comprises, from 5′ to 3′,
a first cistron encoding a light chain of the antibody and comprising a first promoter,
a second cistron encoding a selection marker and comprising a second promoter, and
a third cistron encoding a heavy chain of the antibody and comprising a third promoter,
wherein the first and third promoters are identical or are of substantially equivalent transcriptional strength in a mammalian host cell.

2. The expression construct of claim 1, wherein the recombinant antibody is monospecific.

3. The expression construct of claim 1 or 2, wherein the transposase is a cabbage looper moth transposase.

4. The expression construct of any one of claims 1-3, further comprising a first insulator sequence between the ITR at the 5′ end of the expression cassette (5′ ITR) and the first cistron and/or a second insulator sequence between the third cistron and the ITR at the 3′ end of the expression cassette (3′ ITR).

5. The expression construct of any one of claims 1-4, wherein each of the first and third promoters is a cytomegalovirus (CMV) promoter, a simian virus 40 (SV40) promoter, a truncated SV40 promoter (Δ3SV40), or a hamster β-actin (haBA) promoter, optionally wherein both the first and third promoters are CMV promoters.

6. The expression construct of any one of claims 1-5, wherein the selection marker is glutamine synthetase (GS), optionally wherein the GS is a hamster GS comprising SEQ ID NO:6 or an amino acid sequence at least about 85% identical thereto.

7. The expression construct of any one of claims 1-6, wherein at least one of the cistrons encodes more than one polypeptide.

8. The expression construct of claim 7, wherein the third cistron comprises a coding sequence for a detectable marker upstream of a coding sequence for the heavy chain, wherein
the coding sequence for the detectable marker has a non-ATG start codon and the coding sequence for the heavy chain has an ATG start codon, and
the third promoter directs expression of both the detectable marker and the heavy chain.

9. The expression construct of claim 8, wherein the detectable marker is a cell surface protein, optionally wherein the cell surface protein is a human CD52 comprising SEQ ID NO:8 or an amino acid sequence at least about 90% identical thereto.

10. The expression construct of any one of claims 1-9, wherein the polycistronic expression cassette comprises a ubiquitous chromatin opening element (UCOE), optionally wherein the UCOE
is placed between the second and third cistrons, and/or
comprises SEQ ID NO:9 or a nucleotide sequence at least about 90% identical thereto.

11. The expression construct of any one of claims 1-10, wherein
each of the first and third promoters is a CMV promoter, and
wherein the second cistron comprises a coding sequence for a hamster GS under control of a Δ3SV40 promoter, and/or
the third cistron comprises a coding sequence for a human CD52 upstream of a coding sequence for the heavy chain, wherein the coding sequence for the human CD52 has a non-ATG start codon and the coding sequence for the heavy chain has an ATG start codon, and the third promoter directs expression of both the human CD52 and the heavy chain.

12. The expression construct of claim 11, wherein
the CMV promoter comprises SEQ ID NO:3 or a nucleotide sequence at least about 90% identical thereto,
the hamster GS comprises SEQ ID NO:6 or an amino acid sequence at least about 90% identical thereto,
the Δ3SV40 promoter comprises SEQ ID NO:2 or a nucleotide sequence at least about 90% identical thereto, and/or
the human CD52 comprises SEQ ID NO:8 or an amino acid sequence at least about 90% identical thereto.

13. A method of generating a mammalian host cell that produces a recombinant antibody, comprising:
introducing into a population of mammalian cells (i) the expression construct of any one of claims 1-12 and (ii) the transposase or an expression vector for the transposase,
culturing the cells in a selection medium that allows survival of cells that express the selection marker, and
isolating a surviving cell, thereby generating a mammalian host cell that produces the recombinant antibody.

14. The method of claim 13, wherein the selection marker is GS and the selection medium lacks glutamine.

15. The method of claim 13 or 14, wherein the expression construct expresses a detectable marker and the isolating step comprises selecting a surviving cell that expresses the detectable marker, optionally wherein the detectable marker is a human CD52 and the selecting is by flow cytometry.

16. A mammalian host cell that comprises in its genome the expression construct of any one of claims 1-11, or that is produced by the method of any one of claims 13-15.

17. The mammalian host cell of claim 16, wherein the cell is a CHO cell.

18. The mammalian host cell of claim 17, wherein the CHO cell comprises a null mutation in an endogenous GS gene.

19. A method of producing a recombinant antibody, comprising:
culturing the mammalian host cell of any one of claims 16-18 under conditions that allow expression of the antibody, and
isolating the antibody from the culture.
